(19) United States
(12) Patent Application Publication
(10) Pub. No.: US 2024/0271182 A1
(43) Pub. Date: Aug. 15, 2024

(54) METHODS FOR DETECTING MODIFIED NUCLEOTIDES

(71) Applicant: CAMBRIDGE ENTERPRISE LIMITED, Cambridge (GB)

(72) Inventors: Shankar BALASUBRAMANIAN, Cambridge (GB); Tao YAN, Cambridge (GB)

(21) Appl. No.: 18/534,106

(22) Filed: Dec. 8, 2023

Related U.S. Application Data
(63) Continuation of application No. PCT/EP2022/068096, filed on Jun. 30, 2022.

(30) Foreign Application Priority Data
Jun. 30, 2021 (GB) 2109469.3

(51) Int. Cl. C12Q 1/6806; C12Q 1/6869; G01N 33/58

(52) U.S. Cl. CPC C12Q 1/6806; C12Q 1/6869; G01N 33/58

(57) ABSTRACT

The invention provides a method for identifying a modified cytosine residue, which may be 5-methylcytosine or 5-hydroxymethylcytosine, in a nucleotide sequence. The method comprises oxidising the modified cytosine residue through a non-enzymatic, one-electron process to form 5-formylcytosine. The presence of 5-formylcytosine can be established by labelling and identifying this residue. The invention also provides a method of modifying a polynucleotide containing a 5-methylcytosine and/or a 5-hydroxymethylcytosine residue, a method of oxidising 5-methylcytosine, 5-hydroxymethylcytosine, a 5-methylcytosine residue, or a 5-hydroxymethylcytosine residue, use of a non-enzymatic radical initiator to oxidise a 5-methylcytosine or 5-hydroxymethylcytosine residue, and a kit for use in the methods.

### RELATED APPLICATION

This application is a continuation of International Application No. PCT/EP2022/068096 filed 30 Jun. 2022, which claims the benefit of GB 2109469.3 filed on 30 Jun. 2021 (30 Jun. 2021), the contents of which are hereby incorporated by reference in their entirety.

### SEQUENCE LISTING

The instant application contains a Sequence Listing which has been submitted electronically in XML format and is hereby incorporated by reference in its entirety. Said XML copy, created on Mar. 6, 2024, is named 47224-752_301SL.xml and is 12,946 bytes in size.

### FIELD OF THE INVENTION

This invention relates to the detection of modified cytosine residues and, in particular, to the sequencing of nucleic acids that contain modified cytosine residues. The present invention provides a method of detecting a nucleoside or a nucleotide sequence containing 5-methylcytosine (5mC) or 5-hydroxymethylcytosine (5hmC).

### BACKGROUND

Canonical nucleobases undergo covalent modification in living organisms that introduces chemical functionalities to store epigenetic information in DNA (Bilyard et al.). About 4% of cytosine (C) bases in human DNA are methylated to 5-methylcytosine (5mC), which was coined as the “fifth base” of the human genome (Breiling et al.). The DNA methylation pattern in genomic DNA has an essential role in regulating gene expression, genomic imprinting and X-chromosome inactivation (Schubeler et al.). 5mC has also recently been found to play an essential role in brain signalling (Lister et al.) and aging (Bell et al.).

In metazoa, 5mC can be oxidised to 5-hydroxymethylcytosine (5hmC) by the ten-eleven translocation (TET) family of enzymes (Tahiliani et al.; Ito et al.). 5hmC has been proposed as an intermediate in active DNA demethylation, for example by deamination or via further oxidation of 5hmC to 5-formylcytosine (5fC) and 5-carboxylcytosine (5caC) by the TET enzymes, followed by base excision repair involving thymine-DNA glycosylase (TDG) or failure to maintain the mark during replication (Branco et al.). The 5hmC base may also constitute an epigenetic mark per se.

To map 5mC and 5hmC in genomic DNA is crucial for understanding the biological role of DNA methylation. It is possible to detect and quantify the level of 5mC and 5hmC present in total genomic DNA by analytical methods that include, most notably, bisulfite sequencing (Frommer et al.). Here, the differential activity of unmethylated C relative to 5mC and 5hmC is exploited to allow for detectable changes in certain residues with the amplification and sequencing steps (see Booth et al.; Raiber et al.). The bisulfite sequencing chemistry, as used within TET-assisted bisulfite sequencing (TAB-Seq) and oxidative bisulfite sequencing (oxBS) approaches, is a significant development in the methods for detecting 5mC and 5hmC. Bisulfite sequencing alone does not distinguish between 5mC and 5hmC, and alternative strategies, such as TAB-seq and oxBS-seq, are used to achieve a discrimination between these two modified residues.

The standard approach for identifying DNA methylation (i.e. 5mC) by sequencing uses the bisulfite conversion, where a C to uracil (U) change is effected in a nucleotide sequence, which change is then read as thymine (T) in the subsequent DNA amplification and sequencing.

Limitations of this approach include the reduction of the genetic sequence of each DNA strand to essentially three letters instead of four, which makes it challenging to detect genetic variants: for example all Cs convert to Ts in the sequencing, which makes it impossible to detect C-to-T genetic variants (the most common mutation). Also, bisulfite conversion reduces the complexity of the sequence making it computationally challenging to accurately re-align sequenced reads to the reference genome. Lastly, bisulfite is known to cause some cleavage of DNA at C residues which can cause loss of sequenceable material.

Another way to distinguish 5mC from C is to target the 5-methyl group in 5mC by oxidation. This has been achieved through the use of enzymes, and TET enzymes have been found to recognise and oxidise 5mC to 5hmC, 5fC and 5caC in vitro (Tahiliani et al.; Ito et al.; He et al.).

Current methods of detecting 5mC in vitro that are bisulfite-free rely on TET enzymes to oxidise the 5mC to 5caC. The 5caC can be converted to a uracil analogue by bisulphite treatment (Yu et al.) or pyridine borane reduction (Liu et al. and WO 2019/136413), which can subsequently be read as thymidine (T) during next generation sequencing.

However, a number of drawbacks are associated with the enzymatic detection of 5mC. A multi-stoichiometric quantity of TET enzyme is required to selectively oxidise 5mC to 5caC due to its promiscuous reactivity in vitro (DeNizio et al.). The TET enzyme is also easily degraded and thus requires a complex workflow for sequencing application. Further, TET enzymes have a strong sequence-dependant bias such that these enzymes show very weak in vitro activity on 5mC in a non-CpG context (Hu et al.). Therefore, detection methods that utilise TET enzymes are likely to be biased. Finally, TET enzymes have been reported to show cross-activity by oxidising T to 5-formyluracil (5fU) in vitro (Pais et al.) and are thus not selective for 5mC.

Jonasson et al. describe a method of oxidising the 5-methylcytosine nucleobase using a biomimetic Fe(IV)-oxo complex. This was found to generate a mixture of oxidised products, 5hmC, 5fC, and 5caC. This mixture of products having different reactivities cannot be easily used for downstream functionalisation or sequencing analysis.

A recent work by Jin et al. demonstrated the conversion of monomeric 5-methyldeoxycytidine (5mdC) to 5-formyldeoxycytidine (5fdC) through a photocatalytic pathway. The oxidation reaction was carried out in the presence of DMSO and also required an oxygen atmosphere. These reactions conditions are not compatible with applications on polynucleotides, such as DNA and RNA.

The present inventors have established an alternative method for the detection of 5mC and/or 5hmC in a polynucleotide.

### SUMMARY OF THE INVENTION

In a general aspect the present invention provides a method for oxidising a polynucleotide containing a 5-methylcytosine (5mC) residue and/or a 5-hydroxymethylcytosine (5-hmC) residue. The oxidation product comprises a 5-formylcytosine (5fC) residue.

The oxidation method of the present invention is non-enzymatic and is carried out in the absence of an enzyme, such as a TET enzyme. Enzymatic methods of converting modified cytosine residues in a polynucleotide, such as through use of TET enzymes, can lead to sequence-specific biases, and in particular a bias to modified cytosine residues in a CpG context. Further, TET enzymes may oxidise 5mC or 5hmC residues in a polynucleotide to form 5caC as the major oxidation product, and therefore polynucleotides containing other oxidation products such as 5fC cannot be obtained using this method.

The present inventors have devised methods that allow the modified cytosine residues, 5mC and 5hmC, to be distinguished from canonical cytosine residues. The method can be performed on a nucleobase, or on a nucleoside, a nucleotide, or a polynucleotide comprising 5mC and/or 5hmC residues.

In a first aspect, the invention provides a method of identifying a modified cytosine residue in a sample nucleotide sequence, the method comprising
(i) providing a population of polynucleotides which comprise the sample nucleotide sequence;
(ii) oxidising the modified cytosine residue in the population to form a 5-formylcytosine (5fC) residue through a non-enzymatic, one-electron process;
(iii) labelling the 5-formylcytosine (5fC) residue; and
(iv) identifying the labelled residue within the population,
   wherein the modified cytosine residue is selected from a 5-methylcytosine (5mC) residue and a 5-hydroxymethylcytosine (5hmC) residue.

During step (ii), the modified cytosine residue is oxidised at the carbon that is attached to the C5 position of the pyrimidine ring. The oxidation process forms a 5-formylcytosine (5fC) residue.

In some embodiments, the modified cytosine residue is a 5-methylcytosine (5mC) residue. In other embodiments, the modified cytosine residue is a 5-hydroxymethylcytosine (5hmC) residue.

The one-electron process includes a radical process and involves the generation of a radical. The one-electron process may involve hydrogen atom transfer (HAT) or single-electron transfer (SET).

The aldehyde group of 5fC provides a reactive handle for labelling during step (iii). Methods of functionalising 5fC through the aldehyde group are known in the art, including methods described in Raiber et al., McInroy et al., and US 2020/165661.

Advantageously, the conditions for oxidising the modified cytosines are suitable for use with polynucleotides. The oxidation reaction proceeds in a solvent system in which a polynucleotide is soluble. The reaction conditions, including the reaction temperature and pH, are compatible with polynucleotides and are selected to minimise polynucleotide degradation, such that a substantial amount of polynucleotides can be recovered for downstream analysis following oxidation. This is demonstrated on model oligodeoxyribonucleotides in the examples below.

Accordingly, the present inventors have devised methods that allow 5mC and 5hmC to be selectively targeted in the presence of canonical nucleobases within a polynucleotide. The oxidation product comprises 5fC, which is then labelled in step (iii). The labelled residue can subsequently be detected in step (iv) to identify the modified cytosine residue within the population of polynucleotides. The labelling may be by introduction of a detection tag or an isolation tag. The labelling may convert the 5fC to a residue having a different base-pairing pattern to cytosine, such as a uracil or thymine analogue, which can be subsequently detected by amplifying and/or sequencing the polynucleotide.

The oxidation in step (ii) may be performed in the absence of a TET enzyme, such as in the absence of an enzyme selected from TETI, TET2, and TET3.

Step (ii) may comprise oxidation of the modified cytosine residue in the presence of a radical initiator, to form a 5-formylcytosine (5fC residue). The radical initiator may be a metal-oxo species. In some embodiments, the oxidation in step (ii) may be performed in the presence of a radical initiator that is a photocatalyst, irradiating light, and water, and optionally a single-electron oxidant.

The photocatalyst may have an absorbance maximum in the range 300 nm to 600 nm. That is, the photocatalyst may absorb light in this range to form an excited state. In this way, the oxidation reaction may proceed in the presence of near-ultraviolet (UV) or visible light range and does not require the use of short wavelength UV light (e.g. less than 300 nm), which may damage polynucleotides.

The photocatalyst may be an organic photocatalyst or a transition metal photocatalyst. Preferably, the photocatalyst is a transition metal photocatalyst, and more preferably the photocatalyst comprises a metal-oxo group.

Examples of a photocatalyst include polyoxometalates, such as tungsten polyoxometalates. Preferably, the photocatalyst is selected from decatungstic acid, phosphotungstic acid, and a salt thereof, and more preferably the photocatalyst is decatungstic acid or a salt thereof.

Step (ii) of the method may be performed in the presence of a single-electron oxidant. This helps to accelerate the oxidation reaction, particularly when the oxidation is performed in the presence of a photocatalyst, such that a good yield of 5fC is obtained before substantial degradation of the polynucleotide begins to occur. Preferably, the single-electron oxidant is an organic single-electron oxidant, such as N-fluorobenzenesulfonimide, 5-(trifluoromethyl)dibenzothiophenium tetrafluoroborate, and N-chlorosaccharin.

Step (iii) may comprise labelling the 5fC residue with a detection tag or an isolation tag. A detection tag may comprise a chromophore, a fluorescent label, a phosphorescent label or a radiolabel. An isolation tag may comprise a moiety that binds to a binding agent. The moiety that binds to a binding agent may be biotin. Labelling the 5fC residue in this way allows the polynucleotide comprising the modified cytosine to be identified within the population of polynucleotides, by methods that are well-known in the art.

Preferably, step (iii) comprises labelling the 5fC residue to alter the Watson-Crick base pairing pattern of the 5fC. For example, a nucleophilic probe may be introduced to the 5fC residue to form a derivatised residue having a different base-pairing pattern compared to cytosine. Preferably, the labelled residue is a uracil analogue. Examples of a suitable nucleophilic probe for this labelling include 1,3-indandione and malononitrile. When the 5fC residue is labelled in this way to alter its base-pairing pattern, the position of the 5fC residue may be identified by sequencing of the polynucleotide population.

The labelling of the 5fC residue in step (iii) may comprise deaminating the oxidised residue at the C4 position. Deamination of 5fC forms 5-formyluracil (5fU). The deaminated residue is thus a uracil analogue, and the base-pairing pattern is changed from that for cytosine. This change in base-pairing pattern allows the location of the modified cytosine residue to be identified within the population, such as by sequencing.

The deamination in step (iii) may also be accompanied by reduction of the residue, such as reduction of the pyrimidine ring. The deamination may be performed after the reduction. For example, the 5fC residue may be reduced and then deaminated to form dihydrouracil (DHU). Methods for this transformation are described in WO 2019/136413.

Step (iv) may comprise the steps of:
(a) sequencing the polynucleotides in the population following step (iii) to produce a treated nucleotide sequence; and
(b) identifying the residue in the treated nucleotide sequence which corresponds to a modified cytosine residue in the sample nucleotide sequence.

This allows the location of the modified cytosine residue in the sample nucleotide sequence to be detected by sequencing, such as next-generation sequencing.

The polynucleotide may be DNA or RNA, or a mixture thereof.

The method of oxidising a 5mC or 5hmC residue provides 5fC in good yield. The method is thus advantageous over oxidation methods involving TET enzymes. Typically, TET enzymes oxidise 5mC residues in a polynucleotide to produce a mixture of 5hmC, 5fC and 5caC residues. When the oxidation is performed using a large excess of TET enzymes to the substrate, 5caC is formed as the major oxidation product. For example, Liu et al. report that the oxidation product of 5mC in a polynucleotide using Naegleria TET-like oxygenase (NgTET1) is almost entirely 5caC, with a 5fC yield of only 3%. Thus, the methods of the present invention can also be incorporated into a method of oxidising 5-methylcytosine (5mC) or 5-hydroxymethylcytosine (5hmC) residues in a polynucleotide to form 5fC residues in good yield.

In a second aspect, the invention provides a method of oxidising modified cytosine residues in a sample nucleotide sequence, the method comprising;
(i) providing a population of polynucleotides which comprise the sample nucleotide sequence,
(ii) oxidising the modified cytosine residues in the population to form 5-formylcytosine (5fC) residues, wherein the product mole ratio of 5-formylcytosine (5fC) residues to modified cytosine residues is 10:90 or more,
(iii) optionally labelling the 5-formylcytosine (5fC) residues, and
(iv) optionally identifying the labelled 5-formylcytosine (5fC) residues within the population, wherein the modified cytosine residues are 5-methylcytosine (5mC) residues or 5-hydroxymethylcytosine (5hmC) residues.

The preferred features of the first aspect apply equally to the second aspect.

Preferably, the product mole ratio of 5fC to modified cytosine residues (i.e. either 5mC residues or 5hmC residues) in step (ii) is 20:80 or more, such as 30:70 or more.

The reaction product in step (ii) may be substantially free of oxidation products other than 5fC, such as 5hmC and 5caC. Where the modified cytosine residues provided in the population are 5mC residues, the mole ratio of 5fC product formed in step (ii) to 5hmC and/or 5caC may be 2:1 or higher, such as 5:1 or higher, such as 10:1 or higher, such as 50:1 or higher, such as 100:1 or higher. Where the modified cytosine residues provided in the population are 5hmC residues, the mole ratio of 5fC product formed in step (ii) to 5caC may be 2:1 or higher, such as 5:1 or higher, such as 10:1 or higher, such as 50:1 or higher, such as 100:1 or higher.

In a third aspect, the invention provides a method of modifying a polynucleotide, the method comprising oxidising a 5-methylcytosine (5mC) residue and/or a 5-hydroxymethylcytosine (5hmC) residue in the polynucleotide through a non-enzymatic, one-electron process to form a 5-formylcytosine (5fC) residue.

The preferred features of the first aspect apply equally to the third aspect.

In a fourth aspect, the invention provides a method of oxidising 5-methylcytosine (5mC) or 5-hydroxymethylcytosine (5hmC), the method comprising oxidising the 5-methylcytosine (5mC) or 5-hydroxymethylcytosine (5hmC) through a non-enzymatic, one-electron process to form 5-formylcytosine (5fC).

The preferred features of the first aspect apply equally to the fourth aspect.

In a fifth aspect, the invention provides a method of oxidising a 5-methylcytosine (5mC) residue or a 5-hydroxymethylcytosine (5hmC) residue in a nucleoside, nucleotide or polynucleotide through a non-enzymatic, one-electron process to form a 5-formylcytosine (5fC) residue.

The preferred features of the first aspect apply equally to the fifth aspect.

In a sixth aspect, the invention provides use of a non-enzymatic radical initiator to oxidise a 5-methylcytosine (5mC) residue or a 5-hydroxymethylcytosine (5hmC) residue in a polynucleotide. The radical initiator may be a photocatalyst, which may be used in the presence of irradiating light, water, and optionally a single-electron oxidant.

The preferred features of the first aspect apply equally to the sixth aspect.

In a seventh aspect, the invention provides a kit for use in a method described herein, comprising;
(a) a radical initiator, such as a photocatalyst, such as a polyoxometalate;
(b) a polymerase, and optionally.
(c) a single-electron oxidant, such as an organic single-electron oxidant, such as a compound selected from N-fluorobenzenesulfonimide, 5-(trifluoronnethyl)dibenzothiopheniunn tetrafluoroborate, and N-chlorosaccharin.

These and other aspects and embodiments of the invention are described in further detail below.

### SUMMARY OF THE FIGURES

The present invention is described herein with reference to the figures listed below.

FIG. 1 shows the results of a kinetic study of oxidising a sample comprising equimolar amounts of 5-methyldeoxycytidine, deoxyadenosine, deoxycytidine, deoxyguanosine and deoxythymidine. The solution was oxidised in the presence of 5 mol % Na₄W₁₀O₃₂ and 4 mM NFSI, in a 1:9 mixture of DMSO and water. The sample was irradiated at 365 nm, and the reaction was followed by LCMS over a reaction time of 3 hours.

FIG. 2 shows the sequencing results for a 100mer single-stranded DNA model (5mC-100mer) using the method of the present invention. The signals obtained for 5mC and C positions in the 5mC-100mer are shown. At position 28, which corresponds to 5mC, 32% of reads were observed as thymine, i.e. a 5mC-to-T conversion of 32%. For the remaining positions, essentially all reads were observed as C.

FIG. 3 shows the non-specific mutation rate observed for a 100mer ssDNA (5mC-100mer) using the method of the present invention.

### DETAILED DESCRIPTION OF THE INVENTION

The present invention provides a method for oxidising a polynucleotide containing a 5-methylcytosine (5mC) residue and/or a 5-hydroxymethylcytosine (5-hmC) residue. The oxidation product comprises a 5-formylcytosine residue.

Osberger et al. describe methods of using Fe complexes to selectively oxidise a C—H bond to a carbonyl group in amino acids and peptides by generating an Fe(IV)-oxo species in situ, which is also reviewed in White et al. It is not disclosed that this system can be applied to nucleosides, such as 5-methylcytosine. Also, the use of a strong oxidant such as H₂O₂ to generate the Fe(IV)-oxo species is described, which can degrade nucleosides and polynucleotides such as by depurination.

Jonasson et al. describe a method of oxidising a sample of 5mC to a mixture containing 5hmC, 5fC and 5caC. It is not disclosed that the method is suitable for use on 5mC when present as a residue in a polynucleotide. Further, the mixture of products formed by this method have different functionalities, and cannot be labelled uniformly for downstream analysis in a detection method.

Jin et al. describe a method of converting 5mdC nucleoside to 5fdC nucleoside. The oxidation is carried out in the presence of 90% DMSO and 1 bar oxygen, over a period of 18 hours. This reaction is thus not suitable for carrying out on a polynucleotide, such as DNA, which typically require solvents that are largely aqueous.

Liu et al. describe a method of identifying 5mC (TAPS) that is bisulfite-free and is said to provide resolution at the base level. Here, 5mC and 5hmC are reacted to form 5caC. The method is a two-stage process. In a first step, a 5mC-containing oligomer is treated with a ten-eleven translocation (TET) dioxygenase to form the corresponding 5caC form. In a second step, the 5caC-containing oligomer monomer is treated with a borane to convert the 5caC residue to the corresponding dihydrouracil (DHU). In any subsequent sequencing of the oligomer, the DHU residue is read as T, whereas as the original 5mC residue is read as C.

The TAPS method for generating the 5caC residue involves treatment of a nucleotide sequence containing 5mC with a TET enzyme, and the worked examples demonstrate the use of mTet1CD incubated with a sample nucleotide sequence at 37° C. for 80 minutes. The mixture is then combined with Proteinase K, followed by a further incubation at 50° C. for 60 minutes, and purification to give the oxidised product. The authors note that for “more complete” oxidation, this oxidation procedure should be repeated. The known TET enzymes are TET1, TET2 and TET3.

TET enzymes are known to display a bias towards oxidising methylated or hydroxymethylated cytosine residues in a CpG context. Liu et al. report that the oxidation of residues in a non-CpG context is 11.4% lower than those in a CpG context. Therefore, detection methods that rely on TET enzymes may supress signals from modified cytosine residues in a non-CpG context.

Oxidation by TET enzymes, such as that described in Liu et al., typically convert 5mC and/or 5hmC to 5caC. Whilst 5fC can be formed by TET enzymes, this is usually in trace amounts, which is not enough to be detected in a sequencing method with high confidence. In Liu et al., for example, the yield of 5fC obtained after TET-mediated oxidation is 3%.

Further, the TET dioxygenases are large proteins that can be unstable and difficult to purify.

The prevent inventors have devised a method for oxidising 5mC and/or 5hmC to form 5fC. The oxidation reaction is carried out through a non-enzymatic, one-electron process. Thus, the oxidation reaction does not require the use of enzymes, and in particular does not require the use of TET enzymes.

The oxidation reaction produces 5fC in good yield, without any substantial cross-reactivity observed at canonical cytosine, thymine, adenine or guanine residues. The oxidation product comprises 5fC, and the products of the reaction may be substantially free of other oxidation products, such as 5hmC and 5caC. The aldehyde group in 5fC provides a reactive handle, which can be easily targeted for a labelling reaction. As aldehyde groups are generally absent in biomolecules including polynucleotides, the 5fC obtained by the present methods can be selectively detected by chemical methods.

The reaction can be carried out on a nucleobase, a nucleoside, nucleotide or a polynucleotide. The method is particularly useful for detecting modified cytosine residues in a population of polynucleotides, such as by sequencing.

### Radical Initiator

The methods of the present invention involve the oxidation of 5mC and/or 5hmC at the carbon bonded to the C5 position of the pyrimidine ring. The methods of the invention are believed to proceed via a radical intermediate, which is generated in a one-electron process, using, for example, a radical initiator.

The methods of the present invention therefore provide for the use of a radical initiator to generate radical reactive species for the reaction of 5mC and/or 5hmC.

The radical initiator may be present at a stoichiometric amount, or the radical initiator may be present at an amount that is less than a stoichiometric amount. The radical initiator may also be used a catalyst, which is regenerated during the radical reaction. Here, the catalyst is typically present at less than stoichiometric amount.

The radical initiator may be a metal-oxo species, and/or may be a photocatalyst.

The radical initiator may be a metal-oxo species. A metal-oxo species is a compound having a metal atom that is bonded to an oxygen atom. The metal may be a transition metal, such as a first-row transition metal. Examples include a Fe-oxo compound and a Mn-oxo compound, such as a Fe-oxo compound as described in Osberger et al. A metal-oxo species may further comprise one or more ligands, such as a pyridine, pyrimidine or amine-containing chelating ligand. The ligand may also be selected from those described above.

The radical initiator may not be an enzyme. For example, the radical initiator is not a TET enzyme, for example the radical initiator is not an enzyme selected from TET1, TET2, or TET3.

The one-electron process may comprise a hydrogen atom transfer (HAT), or a single-electron transfer (SET).

The radical initiator may be a photocatalyst. In these embodiments, the one-electron oxidation process in step (ii) is performed in the presence of a photocatalyst, water and incident light. The photocatalyst may optionally be used together with a single-electron oxidant, and preferably it is used so.

By “photocatalyst”, it is meant a radical initiator that is photoinitiated. A photocatalyst is a species that is capable of absorbing light to generate an electron-hole pair (an excited state). Without wishing to be bound by theory, it is thought that the modified cytosine undergoes hydrogen atom abstraction by the photocatalyst at the C5 methyl position to generate a modified cytosine radical. The photocatalyst is believed to selectively abstract a hydrogen atom from the 5-methyl group on 5mC, or from the 5-hydroxymethyl group on 5hmC.

The photocatalyst may absorb light in the near-UV or visible region. Preferably, the photocatalyst has an absorption maximum at 300 nm and above, such as between 300 nm and 600 nm. Irradiation of polynucleotides such as with short wavelength UV, such as below 300 nm, can damage a polynucleotide such as DNA by crosslinking the DNA.

Preferably, the photocatalyst has an absorption maximum in the range 300 to 600 nm, more preferably 300 to 500 nm, and even more preferably in the range 300 to 400 nm.

When step (ii) is performed in the presence of a photocatalyst, the oxidation may comprise irradiating the reaction mixture with light. Typically, the wavelength of light is selected based on the photocatalyst used in the oxidation process. An appropriate light source may be used to illuminate at least part of the reaction mixture.

The photocatalyst may be an organic photocatalyst or a transition metal photocatalyst.

Examples of organic photocatalysts are those based on a ketone, or an acridinium, pyrylium, phenothiazine, phenoxazine, phenazine, phthalonitrile or flavin ring systems. Specific examples include benzophenone, 2,3-butanedione, triphenylpyrylium, 9-Mesityl-10-methylacridiniunn (Mes-Acr), Eosin Y, Fluorescein, riboflavin, riboflavin tetrabutyrate, riboflavin monophosphate and flavin adenine dinucleotide.

Preferably, the photocatalyst is a transition metal photocatalyst.

Examples of transition metal photocatalysts include metal oxides and metal oxide clusters. Metal oxides include WO₃, TiO₂, ZnO, ZrO₂ and metal oxide clusters include TiO₂ clusters.

Transition metal photocatalysts comprising a metal oxide typically also comprise one or more ligands. The ligand may be any ligand that is suitable for stabilising the metal in the transition metal photocatalyst. Where two or more ligands are present, the ligands may be identical (homoleptic) or different (heteroleptic).

Example ligands for transition metal photocatalysts include those based on bipyridine ring systems, phenylpyridine ring systems, bipyrimidine ring systems, bipyrazine ring systems, phenanthroline ring systems and triphenylene ring systems. The ligand may comprise carbon-based conjugated systems which optionally comprise one or more heteroatoms. The transition metal catalyst may comprise cobalt violet (Co₃(PO₄)₂), manganese violet (NH₄MnP₂O₇) or Han Purple (BaCuSi₂O₆).

Preferably, the transition metal photocatalyst comprises a metal oxide cluster. More preferably, the photocatalyst is a polyoxometalate.

Polyoxometalates (POMs) are anionic clusters comprising a transition metal and oxygen atoms. The transition metal in a POM may be an early transition metal, such as vanadium, niobium, tantalum, molybdenum and tungsten. Of these, molybdenum and tungsten are preferred, and tungsten is particularly preferred.

A POM may comprise one type of metal and oxide (an isopolymetalate) or a POM may further comprise a main group oxyanion (heteropolymetalate). The photocatalyst may be doped with a metal or a main group element such as boron, phosphorus or silicone.

Preferably, the POM is selected from a decatungstate (W₁₀O₃₂⁴⁻) and a phosphotungstate (PW₁₂O₄₀³⁻), and the salt forms thereof.

A POM may be provided in the oxidation reaction of the present method in the form of a salt, or as a free acid. Examples of the counterion in the salt include sodium, potassium, and tetrabutylammonium.

Without wishing to be bound by theory, a possible catalytic cycle for the oxidation reaction involving a tungsten-based polyoxometalate is shown in Scheme 1. Two possible pathways for the oxidation of 5mC to 5fC are shown in Schemes 2 and 3. 5-methyldeoxycytidine (5mdC) is shown as an exemplary starting material, however, a corresponding pathway for the oxidation of 5-hydroxymethyldeoxycytidine (5hmdC) is equally plausible.

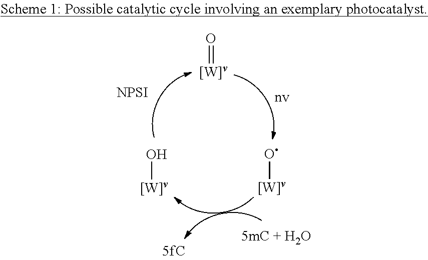

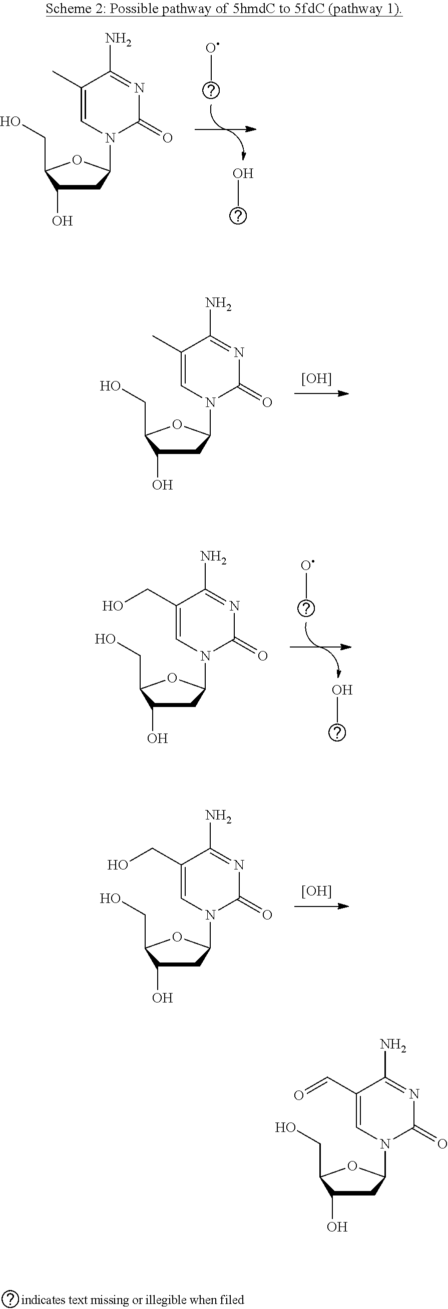

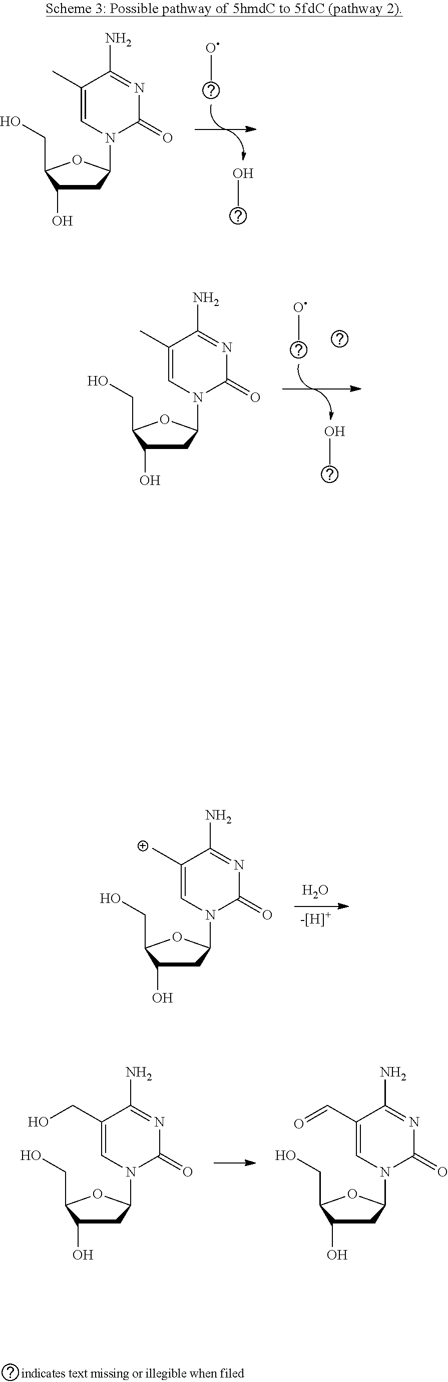

### Single-Electron Oxidant

The oxidation step in the methods of the invention may be carried out in the presence of a single-electron oxidant.

A single-electron oxidant may be capable of accepting an electron from a species through single-electron transfer. In the present case the single-electron oxidant may participate in the oxidation, such as by regenerating the radical initiator in an excited state.

A single-electron oxidant may be an organic species or may be a metal species, which optionally comprises one or more ligands. Preferably, the single-electron oxidant is an organic single-electron oxidant.

Suitable single-electron oxidants include those that may be used in aqueous conditions, which are most convenient for the handling of the polynucleotide. However, single-electron oxidants that are suitable for use in organic solvents may also be used, such as by performing the oxidation reaction in a solvent system comprising an organic co-solvent.

Preferably, the single-electron oxidant is capable of generating one or more radicals selected from
a halogen radical such as a fluoride, chloride or bromide radical;
an oxygen-centred radical such as a peroxide radical;
a carbon-centred radical such as a trifluoromethyl radical;
a nitrogen-centred radical; and
a sulfur-centred radical.

Examples of single-electron oxidants suitable for use in the present invention include the compounds O1, O4, O5, O6, O8 to O13 shown in Scheme 4.

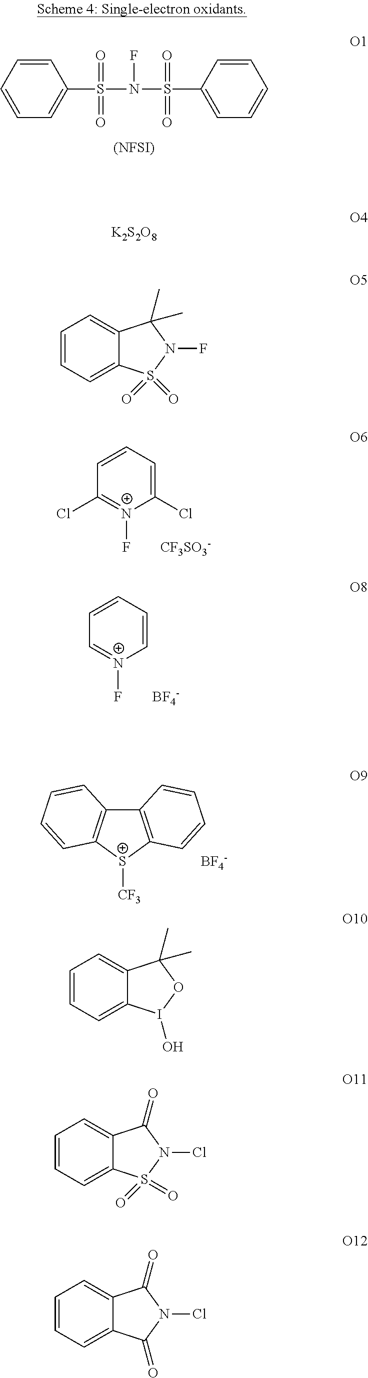

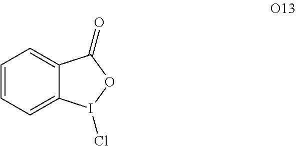 [SMILES: O=C1OI(Cl)c2ccccc21]

Particularly preferred single-electron oxidants include N-fluorobenzenesulfonimide (NFSI), 5-(trifluoromethyl)dibenzothiophenium tetrafluoroborate (O9), and N-chlorosaccharin (O11). These single-electron oxidants accelerate the oxidation reaction whilst reducing the level of degradation of the polynucleotide.

The single-electron oxidant may participate in the oxidation reaction on the modified cytosine residue, and in particular where the oxidation reaction is carried out in the presence of a radical initiator that is a photocatalyst. The single-electron oxidant may accelerate the oxidation reaction. Without wishing to be bound by theory, it is believed that the photocatalyst, in its excited state, generates a radical species from the 5mC or 5hmC residue at the C5 methyl position. The single-electron oxidant may participate in regenerating the ground state of the photocatalyst, as shown in Scheme 1. Isotopic labelling studies in the examples below show that the oxygen atom that is incorporated into the modified cytosine residue is likely to be derived from water. The oxygen atom that is incorporated may also come from molecular oxygen.

### Reaction Mixture and Solvent

The methods of the present case may be undertaken in solution, and this may be an aqueous solution, optionally containing one or more organic solvents.

The method may be performed in a solvent, such as an aqueous solvent. The aqueous solvent may be a mixture of water and one or more organic solvents that are miscible with water.

The oxidation reaction in step (ii) may be carried out in the presence of water, and preferably is done so. The water may be provided by the aqueous solvent.

In one embodiment, the aqueous solvent includes dimethyl sulfoxide (DMSO) or acetonitrile as a co-solvent.

The aqueous solvent system may be an acidic solvent system. The mixture may have a pH in the range pH 3 to less than pH 7, such as pH 4 to less than pH 7, such as pH 4 to pH 6, such as pH 4 to pH 5.

In the present case, a preferred solvent system for use is a water and DMSO mixture at and between about pH 4 and about pH 5.

A buffer may be provided to maintain the pH at a desired level. The buffer may be an acetate, phosphate or ascorbate buffer. The buffer is provided at an appropriate level, as will be clear to a skilled person.

A nucleobase, nucleoside, nucleotide or polynucleotide may be provided in a reaction solvent at an appropriate amount and concentration. These may be present at, for example 1 nM to 1 M.

A nucleoside may be present at a concentration in the range 1 μM to 1,000 mM, such as 0.1 mM to 100 mM, such as 1 mM to 100 mM.

A polynucleotide may be present at a concentration in the range 1 nM to 100 mM, such as 100 nM to 1 mM, such as 1 μM to 100 μM.

The radical initiator, such as a photocatalyst and optionally a single-electron oxidant may each be used at appropriate amounts and concentrations.

The radical initiator may be present at a concentration in the range 1 μM to 100 mM, such as 10 μM to 10 mM.

The single-electron oxidant, where present, may be present at a concentration in the range 100 μM to 5 M, such as 1 mM to 1 M, such as 1 mM to 100 mM.

The methods may be performed at ambient (or room) temperature. For example, the reaction may be performed at a temperature in the range 10 to 25° C.

If necessary, the reaction may be performed at a lower temperature, such as in the range 0 to less than 10° C., or at higher temperature, such as in the range more than 25 to 80° C.

The methods of the present invention may include irradiation of a population of polynucleotides with light of an appropriate wavelength. At least part of the population may irradiated with light. This light may be incident onto all or part of the mixture continuously through the reaction, initially only, or in pulses throughout the reaction, as needed. As described above, the wavelength of light is selected based on the photocatalyst. Any suitable light source may be used to provide the incident light.

A nucleoside or a polynucleotide, such as present within a sample nucleotide sequence, may be treated with a radical initiator, for sufficient time to allow for conversion of 5mC and/or 5hmC to 5fC.

The radical initiator, the optional single-electron oxidant and the reaction conditions during step (ii) may be selected so as to form 5fC as the major reaction product. The reaction may also be repeated, such as by isolating the polynucleotide and repeating step (ii) of the method, to increase the conversion of the 5mC residue.

The oxidation product comprises 5fC. The yield of 5fC obtained at the end of the reaction may be 10% or more, such as 20% or more, such as 30% or more.

The progress of an oxidation reaction may be judged analytically, for example by monitoring the consumption of the starting material nucleoside or polynucleotide and/or monitoring the formation of a reaction product. The reaction may be halted when substantially all of the staring material is consumed, and/or the formation of the product is considered to have a reached a contact maximum. Analytical techniques suitable for reaction monitoring in the present case include UV-vis spectroscopy, LC-MS and NMR spectroscopy.

The reaction for oxidising a modified cytosine with a radical initiator may be at most 24 hours, such as at most 18 hours, such as at most 12 hours, such as at most 6 hours, such as at most 2 hours, such as at most 1 hour. The reaction for oxidising a modified cytosine, may be at least 5 minutes, such as at least 10 minutes, such as at least 30 minutes.

The reaction times may be reduced by, for example, increasing the radical initiator concentration, increasing the single-electron oxidant concentration where present, and decreasing the nucleobase, nucleoside, nucleotide or polynucleotide concentration.

The reaction conditions during oxidation in step (ii) are selected to minimise degradation of the polynucleotide. Some degradation of the polynucleotide, such as 50% of the polynucleotide or less, such as 40% or less, such as 30% or less, may be tolerated. In these embodiments, the amount of the starting material used may be increased so that enough product is obtained following step (ii) for downstream analysis.

After treatment, the treated nucleobase, nucleoside, nucleotide or polynucleotide may be at least partially purified. Here, the product may be separated from the radical initiator and the single-electron oxidant, where present. Techniques for the work-up and isolation of nucleosides, nucleotides and polynucleotides are well known in the art.

Where a method of the invention includes a step for the generation of an oxidised residue from 5mC or 5hmC, that step may be performed in one-pot. Thus, the reaction is undertaken without the isolation or purification of any intermediate forms. Here, pot may broadly refer to a reaction flask, a vial or a well in a well plate, as commonly used in the field of nucleoside preparation and polynucleotide amplification and sequencing.

The sample may also be purified, followed by reintroducing the radical initiator and optionally the single-electron oxidant. In this way, the conversion rate of 5mC may be improved in successive rounds of oxidation.

### Methods

The methods of the invention may be used to oxidise 5mC or 5hmC. The methods may also be used to oxidise a 5mC or 5hmC residue in a nucleoside, nucleotide or polynucleotide.

The invention provides a method for oxidising 5-methylcytosine (5mC) and/or 5-hydroxymethylcytosine (5hmC) to form 5-formylcytosine (5fC) through a non-enzymatic, one-electron process.

The oxidation of 5mC or 5hmC in the methods of the present invention is at the carbon that is bonded to the C5 position of the pyrimidine ring. Thus, the methods involve the oxidation of methyl or hydroxymethyl groups.

The reaction conditions during the oxidation process are suitable for reactions performed on a polynucleotide. Thus, the method may be incorporated in a method for modifying a polynucleotide, the method comprising converting a 5-methylcytosine (5mC) residue and/or a 5-hydroxymethylcytosine (5hmC) residue in the polynucleotide to form a 5-formylcytosine (5fC) residue through a non-enzymatic, one-electron process.

The non-enzymatic, one-electron oxidation process may be performed in the presence of a radical initiator. The radical initiator may be photoinitiated, such as a photocatalyst. An exemplary transformation involving a photocatalyst is shown in Scheme 5, where a 5mC residue in a polynucleotide is oxidised to form a 5fC residue. The oxidation is carried out in the presence of water and light. The reaction is capable of being carried out in air and at ambient temperature, and is therefore conveniently carried out on the polynucleotide substrate.

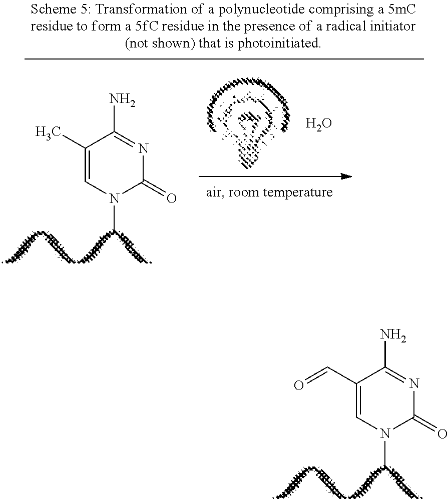

The method of oxidising the 5mC or 5hmC may be incorporated into a method for identifying a modified cytosine residue within a sample nucleotide sequence. Thus, the invention provides a method of identifying a modified cytosine residue in a sample nucleotide sequence, the method comprising
(i) providing a population of polynucleotides which comprise the sample nucleotide sequence;
(ii) oxidising the modified cytosine residue in the population to form a 5-formylcytosine (5fC) residue through a non-enzymatic, one-electron process;
(iii) labelling the 5-formylcytosine (5fC) residue; and
(iv) identifying the labelled residue within the population,
   wherein the modified cytosine residue is selected from a 5-methylcytosine (5mC) residue and a 5-hydroxymethylcytosine (5hmC) residue.

The steps (i) to (iv) above are performed in order.

The methods of the invention are suitable for converting a 5mC or 5hmC residue to a 5fC residue. The methods of the invention therefore provide alternative reaction conditions for this conversion over the methods described in the prior art, including, for example WO 2019/136413.

Methods of identifying a 5fC residue within the polynucleotide in a population of polynucleotides are known in the art. These are described in further detail below, and can be used in steps (iii) and (iv) of the methods above.

The oxidation product formed in step (ii) comprises 5-formylcytosine (5fC) residues. Preferably, the major oxidation product in step (ii) is 5fC. For example, the mole ratio of 5fC residues formed in step (ii) to 5hmC and/or 5caC residues formed may be 2:1 or more, such as 5:1 or more, such as 10:1 or more, such as 50:1 or more, such as 100:1 or more.

The method of oxidising 5mC may be incorporated into a method of identifying 5-methylcytosine (5mC) residues in a sample nucleotide sequence, the method comprising;
(i) providing a population of polynucleotides which comprise the sample nucleotide sequence,
(ii) oxidising the 5-methylcytosine (5mC) residues in the population to form 5-formylcytosine (5fC) residues, wherein the product mole ratio of 5-formylcytosine (5fC) residues to 5-methylcytosine (5mC) residues is 10:90 or more,
(iii) optionally labelling the 5-formylcytosine (5fC) residues, and
(iv) optionally identifying the labelled 5-formylcytosine (5fC) residues within the population.
   The steps (i) to (iv) above are performed in order.

Preferably, the oxidation in step (ii) does not involve the use of an enzyme, such as a TET enzyme. The methods of the present invention advantageously can be used to provide 5fC in good yield, such as where the mole ratio of 5-formylcytosine (5fC) residues products formed after step (ii) to 5-methylcytosine (5mC) residues is 20:80 or more, such as 30:70 or more.

By “product mole ratio”, it is meant the mole ratio of 5-formylcytosine (5fC) residues to modified cytosine residues, such as 5-methylcytosine (5mC) residues, in the product of the oxidation reaction, such as the end of the oxidation reaction.

Step (ii) may optionally comprise purifying the population of polynucleotides after oxidation, such as separating the polynucleotides from the oxidant. In these embodiments, the molar ratio of 5-formylcytosine (5fC) residues to 5-methylcytosine (5mC) residues in the purified population may be 10:90 or more, or as specified above.

The reaction product in step (ii) may be essentially free of alternative oxidation products, such as 5hmC and 5caC. Thus, the product mole ratio of 5fC to 5hmC and/or 5caC residues that is formed in the population may be 2:1 or higher, such as 5:1 or higher, such as 10:1 or higher, such as 50:1 or higher, such as 100:1 or higher. That is, the product mole ratio of 5fC residues to 5hmC residues, to 5caC residues, or to the sum of 5hmC and 5caC ratios is as described above.

The ratios of 5fC to 5hmC and/or 5caC may be determined by, for example, comparison of respective peaks in NMR and LC spectra.

The preferred features of the oxidation reaction, which may include a radical initiator such as a photocatalyst and optionally a single-electron oxidant, and the preferred features of the other steps of the method are as described herein.

The method of oxidising 5hmC may be incorporated into a method of identifying 5hmC residues in a sample nucleotide sequence, the method comprising;
(i) providing a population of polynucleotides which comprise the sample nucleotide sequence,
(ii) oxidising the 5-hydroxymethylcytosine (5hmC) residues in the population to form 5-formylcytosine (5fC) residues, wherein the ratio of 5-formylcytosine (5fC) residues to 5-hydroxymethylcytosine (5hmC) residues is 10:90 or more,
(iii) optionally labelling the 5-formylcytosine (5fC) residues, and
(iv) optionally identifying the labelled 5-formylcytosine (5fC) residues within the population.

Here, the product mole ratio of 5fC to 5hmC residues in step (ii) may be 20:80 or more, such as 30:70 or more. The ratio of 5fC to 5caC formed in step (ii) may be 2:1 or higher, such as 5:1 or higher, such as 10:1 or higher, such as 50:1 or higher, such as 100:1 or higher. Step (ii) may comprise purifying the population of polynucleotides, as described above.

Step (iv) in the methods described herein may comprise the steps of:
(a) sequencing the polynucleotides in the population following step (iii) to produce a treated nucleotide sequence; and
(b) identifying the residue in the treated nucleotide sequence which corresponds to a modified cytosine residue in the sample nucleotide sequence. In these embodiments, the method of identifying a modified cytosine may be a method of sequencing a modified cytosine.

A nucleoside consists of a nucleobase and a sugar. 5mC and 5hmC are examples of a modified, or non-canonical, nucleobase. The sugar may be ribose or deoxyribose.

A nucleotide consists of a nucleoside and a phosphate group. The nucleoside may be as described above.

A polynucleotide, or a nucleic acid, is a polymer comprising nucleotide units. The polynucleotide may be a natural nucleic acid, such as DNA or RNA, or it may be a nucleic acid analogue, such as a peptide nucleic acid (PNA), a phosphorodiamidate morpholino oligomer (PMO), a locked nucleic acid (LNA), a glycol nucleic acid (GNA) or a threose nucleic acid (TNA). The modified cytosine residue may be contained within a mixed nucleic acid comprising any of these elements.

A polynucleotide containing a modified cytosine residue may contain one or more modified cytosine residue i.e. at least one nucleobase is 5mC or 5hmC. For example, a nucleic acid may contain 1, 2, 3, 4, 5 or more modified cytosine residues. One or more modified cytosine residues within a polynucleotide may be labelled using the methods described herein.

The methods of the invention are suitable for use in the analysis of a sample nucleotide sequence. This sample contains a polynucleotide, such as a polynucleotide population, and it may contain a mixture of polynucleotides.

Any sample nucleotide sequence may be an amplified sample. One or more populations may be made of the sample, and each population may be subjected to a different sequencing and identification process. Thus, the methods of the invention may be used in relation to one population to identify a modified cytosine residue in the sample nucleotide sequence, to identify 5mC and/or 5hmC.

In the methods of the invention, a modified polynucleotide is prepared by converting 5mC and/or 5hmC to an oxidised residue including 5fC. The oxidised residue can then be labelled, and the label subsequently detected.

The sample nucleotide sequence may be a genomic sequence. For example, the sequence may comprise all or part of the sequence of a gene, including exons, introns or upstream or downstream regulatory elements, or the sequence may comprise genomic sequence that is not associated with a gene. In some embodiments, the sample nucleotide sequence may comprise one or more CpG islands.

Suitable polynucleotides include DNA, preferably genomic DNA, and/or RNA, such as genomic RNA (e.g. mammalian, plant or viral genomic RNA), mRNA, tRNA, rRNA and non-coding RNA.

The polynucleotides comprising the sample nucleotide sequence may be obtained or isolated from a sample of cells, for example, mammalian cells, preferably human cells.

Suitable samples include isolated cells and tissue samples, such as biopsies, as well as blood samples.

Modified cytosine residues, including 5mC, have been detected in a range of cell types including embryonic stem cells (ESCS) and neural cells (Tahiliani et al.; Itoh et al.; Kriaucionis et al.; Li et al.; Pfaffeneder et al.).

Suitable cells include somatic and germ-line cells.

Suitable cells may be at any stage of development, including fully or partially differentiated cells or non-differentiated or pluripotent cells, including stem cells, such as adult or somatic stem cells, foetal stem cells or embryonic stem cells.

Suitable cells also include induced pluripotent stem cells (iPSCs), which may be derived from any type of somatic cell in accordance with standard techniques.

For example, polynucleotides comprising the sample nucleotide sequence may be obtained or isolated from neural cells, including neurons and glial cells, contractile muscle cells, smooth muscle cells, liver cells, hormone synthesising cells, sebaceous cells, pancreatic islet cells, adrenal cortex cells, fibroblasts, keratinocytes, endothelial and urothelial cells, osteocytes, and chondrocytes.

Suitable cells include disease-associated cells, for example cancer cells, such as carcinoma, sarcoma, lymphoma, blastoma or germ line tumour cells.

Suitable cells include cells with the genotype of a genetic disorder such as Huntington's disease, cystic fibrosis, sickle cell disease, phenylketonuria, Down syndrome or Marfan syndrome.

Methods of extracting and isolating genomic DNA and RNA from samples of cells are well-known in the art. For example, genomic DNA or RNA may be isolated using any convenient isolation technique, such as phenol/chloroform extraction and alcohol precipitation, caesium chloride density gradient centrifugation, solid-phase anion-exchange chromatography and silica gel-based techniques.

In some embodiments, whole genomic DNA and/or RNA isolated from cells may be used directly as a population of polynucleotides as described herein after isolation. In other embodiments, the isolated genomic DNA and/or RNA may be subjected to further preparation steps.

A sample may also be a blood sample, from which circulating free DNA (cfDNA) or circulating tumour DNA (ctDNA) may be extracted.

The genomic DNA and/or RNA may be fragmented, for example by sonication, shearing or endonuclease digestion, to produce genomic DNA fragments. A fraction of the genomic DNA and/or RNA may be used as described herein. Suitable fractions of genomic DNA and/or RNA may be based on size or other criteria. In some embodiments, a fraction of genomic DNA and/or RNA fragments which is enriched for CpG islands (CGIs) may be used as described herein.

The genomic DNA and/or RNA may be denatured, for example by heating or treatment with a denaturing agent. Suitable methods for the denaturation of genomic DNA and RNA are well known in the art.

In some embodiments, the genomic DNA and/or RNA may be adapted for sequencing before treatment, for example before treatment to oxidise a modified cytosine, such as before treatment to oxidise and label a modified cytosine. The nature of the adaptations depends on the sequencing method that is to be employed. For example, for some sequencing methods, primers may be ligated to the free ends of the genomic DNA and/or RNA fragments following fragmentation. In other embodiments, the genomic DNA and/or RNA may be adapted for sequencing after treatment, as described herein.

Following fractionation, denaturation, adaptation and/or other preparation steps, the genomic DNA and/or RNA may be purified by any convenient technique.

Following preparation, the population of polynucleotides may be provided in a suitable form for further treatment as described herein. For example, the population of polynucleotides may be in aqueous solution in the absence of buffers before treatment as described herein.

Polynucleotides for use as described herein may be single-stranded or double-stranded.

The population of polynucleotides may be divided into two, three, four or more separate portions, each of which contains polynucleotides comprising the sample nucleotide sequence. These portions may be independently treated and sequenced, such as described herein.

Preferably, the portions of polynucleotides are not treated to add labels or substituent groups to the modified cytosine residues in a sample nucleotide sequence before treatment, for example before treatment to oxidise the modified cytosine.

### Labelling

Step (iii) of the method comprises labelling the 5fC residue that is formed in step (ii).

The labelling may be to introduce a detection tag to the 5fC residue. A detection tag may include light-sensitive groups such as a chromophore, a fluorescent or a phosphorescent label; or a radiolabel. Such tags are detectable by standard experimental techniques, such as spectroscopic techniques.

The labelling may be to introduce an isolation label to the 5fC residue. An isolation tag may comprise a moiety that binds to a binding agent, such as biotin.

Methods of introducing a tag to 5fC residues in a polynucleotide are known in the art. A tag may be introduced to the formyl group of 5fC, through reaction with a nucleophilic probe. The nucleophilic probe may comprise an amine, hydroxylamine, or hydrazine reactive group. The nucleophilic probe may also comprise a linker to the tag. Examples of introducing an isolation tag to 5fC are described in Raiber et al., McInroy et al., and Hardisty et al.

When the 5fC residue is labelled with an isolation tag, the polynucleotide comprising a modified cytosine residue may be extracted from the population of polynucleotides. These polynucleotides will be labelled via the modified cytosine residue, and may be isolated by contacting the population of polynucleotides with a binding agent, such as an immobilized binding agent. The immobilized binding agents having the labelled polynucleotides bound thereto may be extracted from the population of polynucleotides.

Following extraction, the immobilized binding agents may be washed. Washing removes sample components that are not bound to the binding agent, for example, polynucleotides lacking the labelled residue. Typically, washing procedures include washing with solvents that can remove nucleic acids, such as aqueous buffer.

Following isolation, the polynucleotides containing the labelled residue may be released from the immobilized binding agent. Methods for realising bound substrates are well known in the art.

The labelling may be to introduce a mutation to a 5fC residue formed in step (ii). By “mutation”, it is meant a hydrogen-bonding pattern on the Watson-Crick (N3-C4) face of the modified cytosine residue that differs from the hydrogen bonding pattern typically observed for cytosine residues, such that the modified cytosine residue base-pairs with a nucleobase other than guanine during a polymerase chain reaction (PCR). Typically, the mutation will be a C to T mutation, such that during PCR amplification, copies of the polynucleotide are generated where the modified cytosine residues within the polynucleotide are replaced with a thymine residue. Methods of introducing a mutation to a 5fC residue are known. Examples include reacting a 5fC with a nitrile compound or an 1,3-indandione compound as described in US 2020/0165661 and in Xia et al. as well as reducing 5fC to form DHU, such as by a borane as described in Liu et al.

Preferably, the labelling in step (iii) comprises converting the 5fC residue to a uracil analogue. This may be by reacting the 5fC with a nitrile compound, such as malononitrile, to form a bicyclic nucleobase residue that base-pairs with adenine during PCR amplification of the polynucleotide. The location of the modified cytosine residue may then be identified by sequencing, as a C-to-T mutation.

The population of polynucleotides comprising a sample nucleotide sequence may be first divided into two or more portions in the method of the present invention. The method, comprising steps (i) to (iv), may be performed on a first portion, wherein step (iii) comprises the converting the 5fC residue to a uracil analogue. The first portion is then sequenced by conventional methods. A second portion is also sequenced, without performing the step (ii) and/or (iii). The location of the modified cytosine residue within a polypeptide may then be identified by comparing the sequencing reads, such as by detecting a C-to-T mutation. The detection may thus be by sequencing the polynucleotides in the population to produce a treated nucleotide sequence, followed by identifying the residue in the treated nucleotide sequence which corresponds to the modified cytosine residue in the sample nucleotide sequence.

### Sequencing

The polynucleotides may be adapted after treatment to be compatible with a sequencing technique or platform. The nature of the adaptation will depend on the sequencing technique or platform. For example, for Solexa-Illumina sequencing, the treated polynucleotides may be fragmented, for example by sonication or restriction endonuclease treatment, the free ends of the polynucleotides repaired as required, and primers ligated onto the ends.

Polynucleotides may be sequenced using any convenient low or high throughput sequencing technique or platform, including Sanger sequencing, Solexa-Illumina sequencing, Ligation-based sequencing (SOLiD™), pyrosequencing; strobe sequencing (SMRT™); semiconductor array sequencing (Ion Torrent™); and nanopore sequencing (ION).

Suitable protocols, reagents and apparatus for polynucleotide sequencing are well known in the art and are available commercially.

The residues at positions in the first and other sequences which correspond to cytosine in the sample nucleotide sequence may be identified.

When the 5fC residue is labelled in step (iii) of the method to introduce an isolation tag, the identity of the original modified cytosine residue can be determined by extracting the polynucleotides comprising the isolation tag from the population of polynucleotides, followed by sequencing of the extracted polynucleotides. Preferably, the population of polynucleotides is divided into at least two portions. The steps (i) to (iv) of the method of the present invention is performed on a first portion (an enriched portion), and a second portion is left untreated (a control portion). Sequencing of the two portions and comparing the sequencing reads allows the identity of the polynucleotides containing the original modified cytosine residue to be identified. Methods for carrying out enrichment sequencing in this way are described in the art, such as Raiber et al. and Hardisty et al.

As described above, when the 5fC residue is converted in step (iii) to a uracil analogue, the location of the modified cytosine residue within a polynucleotide may be determined by sequencing the polynucleotide sample. Where the sequence of the polynucleotide is known, the location of the modified cytosine residue within the sample nucleotide sequence can be identified by comparison with the known sequence. Where the sequence of the polynucleotide is unknown, the sequencing reads can be compared to those obtained for a portion of the polynucleotide that has not undergone the oxidation (i.e. step (ii)) and/or the labelling (i.e. step (iii)). Thus, the methods of the invention may enable the modified cytosine residue to undergo a C-to-T transition such as during amplification, which can be detected by conventional sequencing methods.

The extent or amount of cytosine modification in the sample nucleotide sequence may be determined. For example, the proportion or amount of 5mC or 5hmC in the sample nucleotide sequence compared to unmodified cytosine may be determined.

Polynucleotides as described herein may be immobilised on a solid support.

A solid support is an insoluble, non-gelatinous body which presents a surface on which the polynucleotides can be immobilised.

Examples of suitable supports include glass slides, microwells, membranes, or microbeads. The support may be in particulate or solid form, including for example a plate, a test tube, bead, a ball, filter, fabric, polymer or a membrane. Polynucleotides may, for example, be fixed to an inert polymer, a 96-well plate, other device, apparatus or material which is used in a nucleic acid sequencing or other investigative context. The immobilisation of polynucleotides to the surface of solid supports is well-known in the art. In some embodiments, the solid support itself may be immobilised. For example, microbeads may be immobilised on a second solid surface.

In some embodiments, the first and/or second portions of the population of polynucleotides may be amplified before sequencing. Preferably, the portions of polynucleotide are amplified following oxidation and labelling.

Suitable methods for the amplification of polynucleotides are well known in the art.

Following amplification, the amplified portions of the population of polynucleotides may be sequenced.

Nucleotide sequences may be compared and the residues at positions in the first and second nucleotide sequences which correspond to modified cytosine in the sample nucleotide sequence may be identified, using computer-based sequence analysis.

Nucleotide sequences, such as CpG islands, with cytosine modification greater than a threshold value may be identified. For example, one or more nucleotide sequences in which greater than 1%, greater than 2%, greater than 3%, greater than 4% or greater than 5% of cytosines are 5-methylated and/or 5-hydroxymethylated may be identified.

Computer-based sequence analysis may be performed using any convenient computer system and software. A typical computer system comprises a central processing unit (CPU), input means, output means and data storage means (such as RAM). A monitor or other image display is preferably provided. The computer system may be operably linked to a DNA and/or RNA sequencer.

The methods of the invention allow for this modified polynucleotide to be compared against a polynucleotide sequence that is not treated. A comparison between these sequences can show where there has been a C to T change upon treatment. Thus, the presence of 5mC and/or 5hmC may be determined.

Thus, a sample nucleotide sequence may include an untreated portion and a treated portion. The polynucleotides in each portion may be sequenced, and compared against each other to allow for identification of a modification in the treated portion.

In the methods of the present case, any step of identifying a modified cytosine in a sample includes the step of treating a population of a nucleotide sample, such that 5mC and/or 5hmC residues within a polynucleotide are converted to 5fC residues. The treated polynucleotide may be sequenced and the residue in the treated nucleotide sequence which corresponds to a modified cytosine residue in the sample nucleotide sequence may be identified. Here, identification may follow a change in sequenced residues between the sample and the treated polynucleotides. Thus, 5mC and 5hmC, which are read as C, are read as T in the treated sequence. Thus, the presence of a thymine residue in the treated nucleotide sequence is indicative that the modified cytosine residue in the sample nucleotide sequence is 5mC or 5hmC.

Thus, in one embodiment of the invention, a sample nucleotide sequence may be made into two or three populations. A first population may be analysed using the methods of the invention. Thus, a 5mC or 5hmC residue in a polynucleotide may be oxidised to a 5fC residue. The resulting polynucleotide may then be sequenced and the modified cytosine residue identified in the usual way. This method may be combined with the methods described below for a second population.

A second population may be treated with a protecting agent, to protect a 5hmC residue in a polynucleotide, for example as glucose-protected 5-hydroxymethylcytosine (5gmC). The treated population may then be subsequently further treated to convert a 5mC residue in a polynucleotide to a 5fC residue, and then this 5fC residue to a labelled residue. The resulting polynucleotide may then be sequenced and the modified cytosine residue identified in the usual way.

A third population may be treated with a blocking agent to convert pre-existing 5fC residues in a polynucleotide to a species that is not reactive to the labelling reaction in step (iii). The blocking agent may be a nucleophile, such as a hydroxylamine or a hydrazine. The resulting polynucleotide may then be sequenced and the modified cytosine residue identified in the usual way.

An analysis of a sample nucleotide sequence with multiple populations is described, for example, by Liu et at. and WO 2019/136413. The methods for transforming 5mC, 5hmC and 5fC, and the accompanying methods of analysis, disclosed in these documents are incorporated by reference herein.

### Uses

In a further general aspect, the invention provides the use of a non-enzymatic radical initiator to oxidise a 5mC residue and/or a 5hmC residue in a polynucleotide. The oxidation involves a one-electron process.

Thus, the invention provides use of a radical initiator, to convert a 5mC residue and/or a 5hmC residue in a polynucleotide to form a 5fC residue. The radical initiator may be a photocatalyst and the use may be in the presence of light, water, and optionally a single-electron oxidant.

The preferred features of the radical initiator, reaction conditions and reaction products are as described herein.

### Kits

In a further aspect the invention provides a kit comprising:
(a) a radical initiator as described herein;
(b) a polymerase; and optionally
(c) a single-electron oxidant as described herein.

The kit may be provided in a suitable container and/or with suitable packaging.

The polymerase may be a DNA polymerase or an RNA polymerase. The polymerase may be a thermostable polymerase, for example a high discrimination polymerase. Preferably, the polymerase is a uracil-tolerant polymerase and is capable of DNA synthesis past a labelled cytosine residue.

Optionally, the kit may include instructions for use, e.g., written instructions on how to use the kit in a method of detecting 5mC in a polynucleotide sample.

A kit may further comprise a population of control polynucleotides comprising one or more modified cytosine residues, for example cytosine (C), 5-methylcytosine (5mC), 5-hydroxymethylcytosine (5hmC) or 5-formylcytosine (5fC). In some embodiments, the population of control polynucleotides may be divided into one or more portions, each portion comprising a different modified cytosine residue.

The kit may include instructions for use in a method of identifying a modified cytosine residue as described above.

A kit may include one or more other reagents required for the method, such as buffer solutions, sequencing and other reagents. A kit for use in identifying modified cytosines may include one or more articles and/or reagents for performance of the method, such as means for providing the test sample itself, including DNA and/or RNA isolation and purification reagents, and sample handling containers (such components generally being sterile).

A kit may include sequencing adapters and one or more reagents for the attachment of sequencing adapters to the ends of isolated nucleic acids, such as T4 ligase.

A kit may include one or more reagents for the amplification of a population of nucleic acids using the amplification primers. Suitable reagents may include dNTPs and an appropriate buffer.

### Other Embodiments

Each and every compatible combination of the embodiments described above is explicitly disclosed herein, as if each and every combination was individually and explicitly recited. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### Results and Discussion

The methods of the invention were exemplified on both nucleosides and oligodeoxyribonucleotides (ODNs).

### Materials and Methods

Reagents were obtained from Sigma-Aldrich, Acros, or Alfa Aesar, and used without further purification. Enzyme solutions were obtained from Zymo, New England BioLabs, or Sigma-Aldrich and used directly. Na₄W₁₀O₃₂ was synthesized based on a literature procedure (Sarver et al.).

Oligodeoxyribonucleotides (ODNs), including short ODNs for reactions, 100mer ss-DNA strands and template and primers were custom synthesised and HPLC-purified by ATDBio or Sigma-Aldrich and used without further purification after dissolution into ultrapure H₂O (Milli-Q H₂O, purified by Milli-Q Type 1 Ultrapure Water Systems, Merck).

Sequences of model ODNs are shown in Table 1.

TABLE 1
Model ODN sequences

| ODN | Sequence |
|---|---|
| 5mC-13 mer | 5′-ACGT[5mC]ACGTACGT-3′ |
| 5mC-24 mer | 5′-CG[5mC]GTTAACGTTAACGTTAACGCG-3′ |
| 5mC-100 mer | 5′-TCAGGTCTGGAGCAATGCAGCAGTTCG[5mC]GCGCATGCT |
| | TAACAGTACAGATTTCGGATCCATCGTTATCGCTCTGAAAGT |
| | ACAGATCCTCAGTGGTTGGCT-3′ |
| 5fC-100 mer | 5′-TCAGGTCTGGAGCAATGCAGCAGTTCG[5fC]GCGCATGCTTA |
| | ACAGTACAGATTTCGGATCCATCGTTATCGCTCTGAAAGTACAG |
| | ATCCTCAGTGGTTGGCT-3′ |
| Primer | 5′-AGCCAACCACTGAGGATCTG-3′ and |
| | 5′-TCAGGTCTGGAGCAATGCAG-3′ |

LC-MS spectra were recorded on an Amazon X ESI-MS (Bruker) connected to an Ultimate 3000 LC (Dionex). Single deoxyribonucleotides were analysed on a Waters Acquity premier HSS T3 column (1.8 μm, 2.1×100 mm, part No. 186009471) (Method: eluent A, 5 mM NaHCO3 aqueous solution; eluent B, MeCN. Flow rate, 0.5 mL/min. Pre-wash for 12 minutes with 2% eluent B; 8 minutes, 2% eluent B; 1 minute, gradient of 2-60% eluent B; 5 minute, 60% eluent b; 1 minute, gradient of 60-5% eluent B).

ODNs were analysed using a gradient of 5-30% or 5-40% methanol vs. an aqueous solution of 10 mM triethylamine and 100 mM hexafluoro-2-propanol on a Waters XBridge Oligonucleotide BEH C18 column (130 Å, 2.5 μm, 2.1×50 mm) or Acquity Premier Oligonucleotide BEH C18 column (130 Å, 1.7 μm, 2.1×50 mm) (with 0.5 mL/min flow rate for 10-15 minutes). Mass chromatograms shown are base peak chromatograms, UV absorption was recorded at 260 nm.

High-resolution mass spectra (HRMS) of ODNs were conducted on a Shimadzu LC-MS 9030 QToF using a gradient of 5-30% methanol vs. an aqueous solution of 10 mM triethylamine and 100 mM hexafluoro-2-propanol on a XTerra MS C18 column (125 Å, 2.5 μm, 2.1×50 mm) with TMS endcapping.

Reactions were carried out under air unless otherwise stated. Reactions were monitored by LC-MS.

Photo reactor (HCK1006-01-016) and lamp (HCK1012-01-006, 365 nm, 30 W) was purchased from HepatoChem (Beverly, MA 01915 USA).

All photocatalytic reactions were performed in 2 mL glass vials. An O2-free reaction was performed in a 20 mL Schlenk tube.

Automated gel electrophoresis was performed using an Agilent Technologies 2200 Tapestation, D1000 ScreenTapes and sample buffer.

Oligo were purified by Zymo Oligo Clean & Concentrator Kits (D4060) using the supplier's protocol (iPrOH was used instead of EtOH).

PCR samples were purified by Thermo Fisher GeneJET PCR Purification Kit following the supplier's protocol.

DNA sequencing sample libraries were prepared using NEBNext Ultra II DNA Library Prep Kit for Illumina (E7645S), indexed with NEBNext Multiplex Oligos for Illumina (E6609S), sequenced with Illumina MiSeq Reagent Nano Kit v2 (300-cycles) (MS-103-1001), in an Illumina MiSeq sequensor.

### General Methods

### General Procedure A: Selective Oxidation of 5mdC to 5fdC.

To a 2 mL transparent glass vial, stock solutions of monodeoxynucleosides, Na₄W₁₀O₃₂ and other reagents as specified were added. The mixture was diluted with H₂O plus organic solvent where specified, to the targeted concentration and volume. The vial was capped under air. It was then placed in the photoreactor under irradiation at 365 nm for the specified period of time. At the end of the reaction, the mixture was diluted by water and analysed by LC-MS.

### General Procedure B: O₂-Free Selective Oxidation of 5mdC to 5fdC (Freeze-Pump-Thaw).

To a 20 mL Schlenk tube, stock solution of single deoxyribonucleotides, Na₄W₁₀O₃₂ and other reagents where specified were added. The mixture was diluted with H₂O plus organic solvent where specified, to the targeted concentration and volume. The Schlenk tube was capped and placed in the liquid nitrogen. Once the solution was completely frozen, the Schlenk tube was connected to high vacuum through a Schlenk double-line. A vacuum-argon cycle was performed for three times before the solution was melted at room temperature. The freeze-pump-thaw procedure was repeated. The Schlenk tube was then placed in the photoreactor under irradiation at 365 nm for the specified period of time. At the end of the reaction, the mixture was diluted by water and analysed by LC-MS.

### General Procedure C: Selective Oxidation of 5mC to 5fC in Oligodeoxyribonucleotides.

To a 2 mL transparent glass vial, stock solutions of ODNs, Na₄W₁₀O₃₂, NFSI and other reagents as specified were added. The mixture was diluted with H₂O plus organic solvent where specified to the targeted concentration and volume. The vial was capped under air. It was then placed in the photoreactor under irradiation at 365 nm for the specified period of time. At the end of the reaction, the reaction was purified by Zymo oligo concentrator and ODNs were analysed by LC-MS.

General Procedure D: Conjugation on Converted Oligo with (+)-Biotinamidohexanoic Acid Hydrazide.

The photochemically converted oligo was purified and diluted with water to 70 μL. To the solution, 10 μL of 100 mM hydrazide in DMSO, 10 μL of 1 M p-anisidine in MeOH and 10 μL of 400 mM NH₄OAc (pH=5) were added. The mixture was stirred at 25° C. for 20 hours before purification using a Zymo oligo concentrator. The purified oligo was analysed by LC-MS.

General Procedure E: Conjugation on Converted Oligo with Malononitrile.

The photochemically converted oligo was purified and diluted with water to 60 μL. To the solution, 40 μL of 1 M malononitrile in water was added. The mixture was stirred at 25° C. for 20-24 hours before purified by Zymo oligo concentrator. The purified oligo was analysed by LC-MS.

Photocatalytic Conversion of 5mC-13mer, Followed by Enzymatic Digestion.

A 100 pL solution (V_{DMSO}/V_{H2O}=1/9), contained 10 μM 5mC-13mer, 50 μM Na₄W₁₀O₃₂ and 10 mM NFSI, in a 2 mL glass vial, which was stirred under 365 nm irradiation (30 W) for 30 minutes at 20-25° C. At the end of the reaction, it was purified by a Zymo oligo concentrator and purification kit immediately. The purified ODN was solubilised in ultrapure H₂O and analysed by LC-MS. The sample of the ODN (90%) was subjected to enzymatic digestion by Zymo DNA degradase plus. A 50 μL solution of ODN with DNA degradase buffer provided by the supplier and 10 U DNA degradase was incubated at 37° C. for 5 hours. The digested sample was purified by a pre-washed (400 μL ultrapure H₂O) Amicon Ultra-0.5 ml 10K centrifugal filter (Merck) and washed on the filter with additional 40 μL ultrapure H₂O. The purified solution was analysed by LC-MS.

Sequencing the 5mC Site in 5mC-100mer by NGS.

A 100 μL solution (V_{DMSO}/V_{H2O}=1/9), contained 5 μM 5mC-100mer, 20 μM Na₄W₁₀O₃₂ and 10 mM NFSI, in a 2 mL glass vial, which was stirred under 365 nm irradiation (30 W) for 1-2 hours at 20-25° C. At the end of the reaction, it was purified by an oligo purification kit immediately (this reaction can be repeated to increase the conversion of 5mC). The purified oligo was stirred in 100 μL of 400 mM malononitrile aqueous solution at 25° C. for 24 hours before purified by a Zymo oligo concentrator and purification kit. A small portion of the purified oligo was amplified by PCR using Taq Hot Start polymerase (NEB). The PCR product was validated on an Agilent 2200 TapeStation using a D1000 ScreenTape. It was then purified by a Thermo Fisher GeneJET PCR purification kit. The purified PCR product was used to prepare a sequencing library using a NEBNext Ultra II DNA Library Prep kit, and indexed by NEBNext Multiplex Oligos. To the NaOH (aq.) denatured library, an equal molar amount of denatured PhiX solution was added to provide 6 pM end concentrations of the library (following the supplier's protocol). The library was sequenced using an in-house Illumina MiSeq sequencer with a MiSeq Reagent Nano Kit v2. The data was analysed through a customised pipeline.

PCR conditions are shown in Table 2.

TABLE 2
PCR conditions

| | | |
|---|---|---|
| Reagent | Final c/amount | Amount to add |

| | | | |
|---|---|---|---|
| 10x reaction buffer | 1X | 40 | μL |

| | | | | |
|---|---|---|---|---|
| 100 mM dNTPs | 200 | μM | 0.8 | μL *4 |
| 100 μM forward primer | 0.5 | μM | 2 | μL |
| 100 μM reverse primer | 0.5 | μM | 2 | μL |
| Template oligo 1 nM 100mer | | | 10 | μL |
| H₂O | | | 340 | μL |
| Dream Taq polymerase | 10 | U | 2 | μL |

The 400 μL solution was split into 8 tubes with 50 μL each.

PCR reactions were performed on a T100 Thermocycler (BioRad). Method: Lid, 105° C.; step 1, 95° C., 2 min; step 2, 95° C., 30 s; step 3, 62° C., 30 s; step 4, 72° C., 1 min; step 5, go to step 2, repeat 40 times; step 6, 72° C., 1 min; step 7, infinite hold at 12° C.

### Oxidation of Nucleosides

A tungstate-based polyoxometalate, sodium phosphotungstate (Na₃PW₁₂O₄₀), was found to promote efficient conversion of 5mdC to 5fdC. At 100 μM concentration, 100 μM Na₃PW₁₂O₄₀, 20% DMSO in water, under 365 nm light-irradiation for 18 hours, 5mdC can be fully converted to 5fdC with >95% selectivity (Scheme 6).

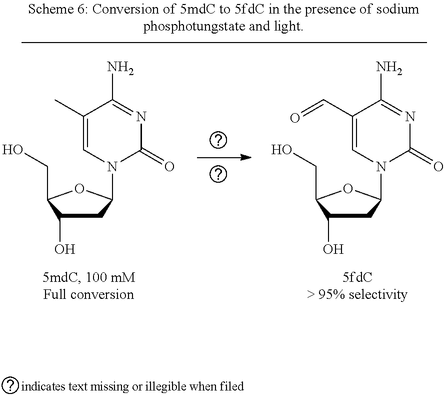

The combination of a polyoxometalate with a single-electron oxidant was also investigated. No significant improvement was observed after testing different types of oxidants with sodium phosphotungstate.

Surprisingly, when sodium decatungstate (Na₄W₁₀O₃₂) was used as the catalyst (Sarver et al.), a significant acceleration was observed when combined with a single-electron oxidant (Scheme 7). The oxidants N-fluorobenzenesulfonimide (NFSI), 5-(trifluoromethyl)dibenzothiophenium tetrafluoroborate (O9), and N-chlorosaccharin (O11) each gave 53-70% conversion of 5mdC in 2 hours. In contrast, poor conversion was observed when H₂O₂ was employed instead. The results for reactions that use a single-electron oxidant selected from compounds O1 to O13 are shown in Table 3. Reactions were carried out according to the General Procedure A for 2 hours, using 5 mM 5mdC in 10% DMSO in water and 5 mol % sodium decatungstate as the photocatalyst unless stated otherwise.

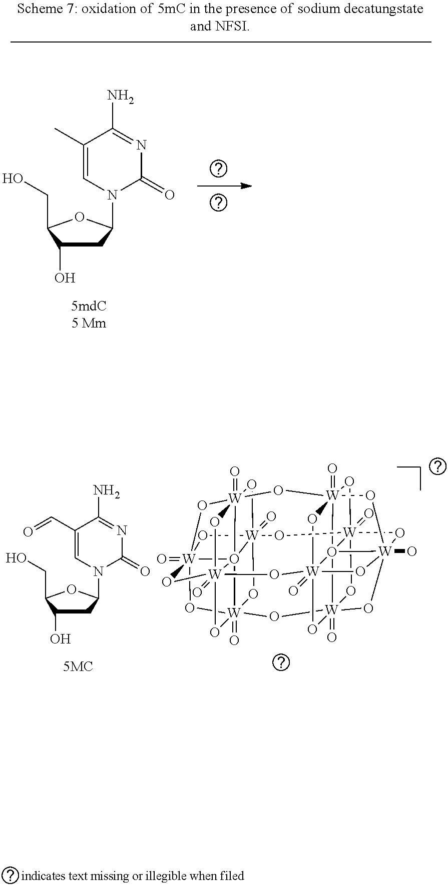

TABLE 3
conversion of 5 mdC in the presence of sodium
decatungstate and a single-electron oxidant.

| Oxidant | Conversion of 5 mdC |
|---|---|
| O1 | 70% |
| O2 | _{<5}% |
| O3 | <5% |
| O4 | 67% |
| O5 | 26% |
| O6 | 41% |
| O7 | <1% |
| O8 | 24% |
| O9 | 66% |
| O10 | 15% |
| O11 | 53% |
| O12 | 28% |
| O13 | 45% |
| H₂O₂ | <5% |
| O1 in 50% DMSO | <5% |
| O1 with 25 mol % Na₃PW₁₂O₄₀ | <5% |

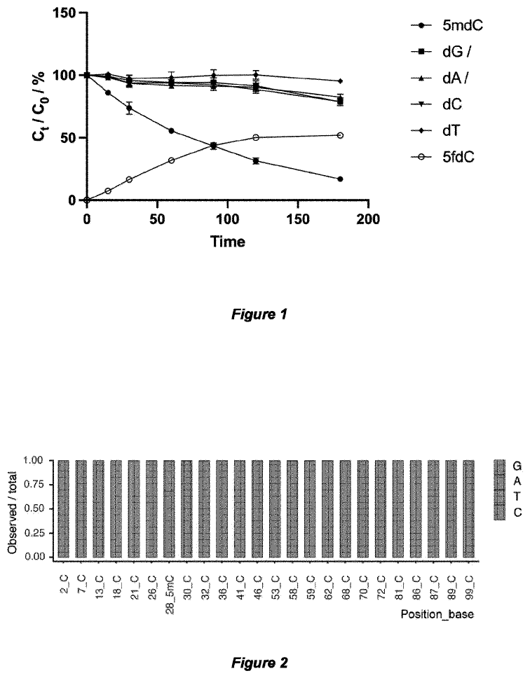

For the reaction with sodium decatungstate and NFSI, a kinetic study was carried out (Scheme 8). To mimic the molecular functionalities of DNA, an equal molar mixture of 5mdC, deoxyadenosine (dA), deoxycytidine (dC), deoxyguanosine (dG) and deoxythymidine (dT) were used for this study. After 2 hours, 70% of 5mdC was converted to 5fdC with >70% selectivity (FIG. 1). The concentrations of dA, dC, dG and dT were very stable over the 2-hour reaction. Running the reactions for the extended hours, increasing amount of side reactions were observed including cross-active C—H oxidation and depurination.

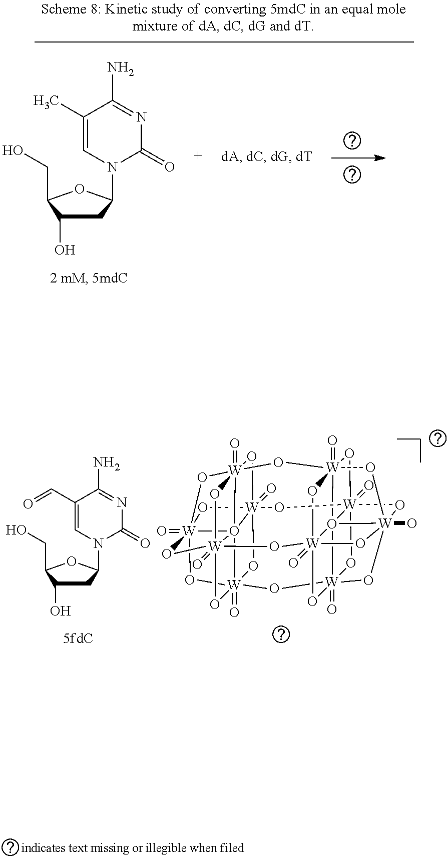

To better understand the reaction, several control experiments were performed. When the reaction was carried out in argon, the outcome was comparable as in air (Scheme 9). After 3 hours, 71% of 5mdC was converted with 58% yield of 5fdC. When the reaction was performed in H₂O with enriched ¹⁸O, ¹⁸O labelled 5-hydroxymethyldeoxycytidine (5hmdC) was detected as an intermediate. This suggests that the oxygen source in the oxidation is H₂O. Hong et al. have also disclosed a photocatalytic C—H oxidation process that is believed to use H₂O as the oxygen source.

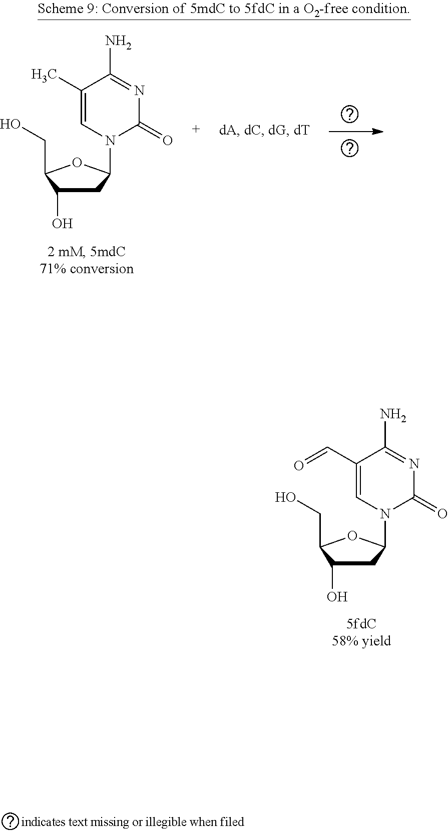

Compound 5hmdC was also found to be oxidised to 5fdC under the same condition (Scheme 10). At 3 hours, 77% of 5hmdC was converted with 60% yield of 5fdC.

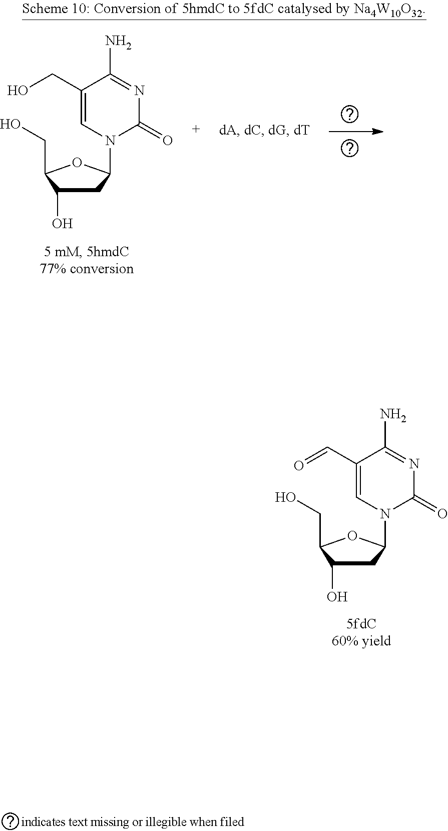

### Oxidation of Oligodeoxyribonucleotides

Next, 5mC residue in ODNs were oxidised (Scheme 11). A 13mer ODN containing one 5mC residue (5mC-13mer) was used.

In the context of ODNs, a 10% conversion of 5mC residues to 5fC residues was observed using sodium phosphotungstate (Scheme 10). However, sodium phosphotungstate was found to largely degrade the ODN before a significant conversion of the 5mC residue. This is likely due to the cleavage of the phosphodiester backbone (Han et al.). Therefore, a large amount of starting material is required to recover useful amounts of the oxidation product using this radical initiator.

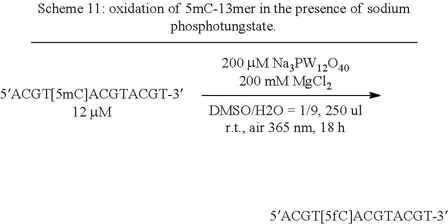

When oxidation was carried out in the presence of sodium decatungstate and NFSI (O1), 20-40% conversion of the 5mC to 5fC was observed after a 15-30 minute reaction (based on the mass change of the purified ODN) (Scheme 12). About 30 mol % loss of the ODN starting material was also observed based on UV spectroscopy. No significant amount of depurination was observed.

When oxidation was carried out in the presence of sodium decatungstate and either (O6) or N-chlorosaccharin (O11), around 10% conversion of the 5mC to 5fC was observed within 15-30 minutes.

The 5mC-13mer ODN and the oxidised ODN could be detected by LC-MS after the reaction was carried out, confirming that the ODN was not largely degraded during the course of the reaction and can be recovered.

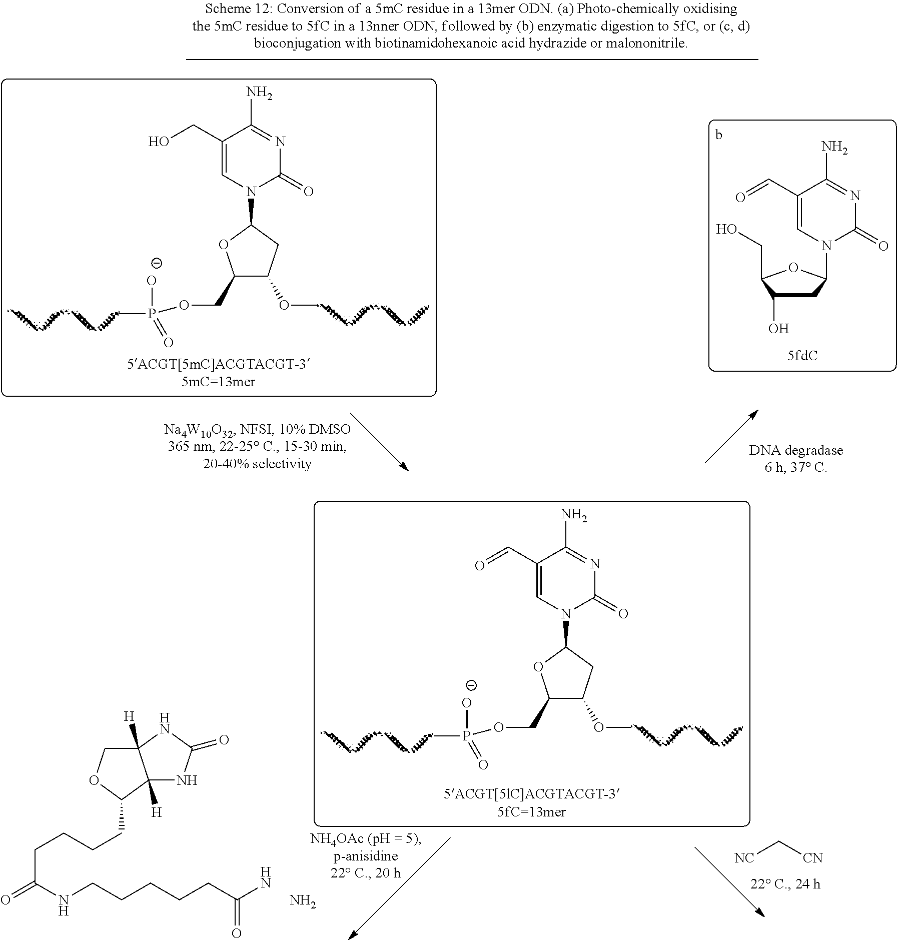

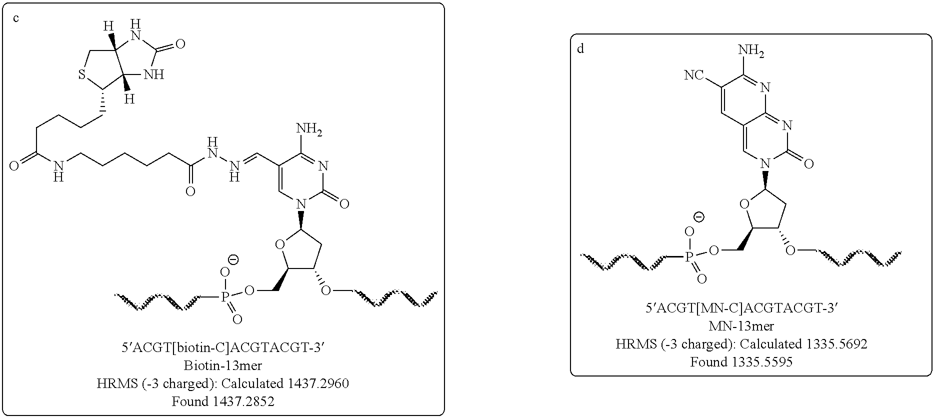

The initial discovery of 5fC in the genome was relied on mass spectrometry study of the digested DNA, according to the method described in Pfaffeneder et al. In the present study, to confirmed 5fC residue was obtained after the photochemical oxidation of the 5mC residue, the converted ODN was purified and digested to single nucleoside. The 5fC trace was indeed observed in the digested mixture, thus confirming the formation of this residue. No 5-formyldeoxyuridine (5fdU) trace was observed in the digested mixture. A self-complimentary 24mer ODN (5mC-24mer) was also investigated for the reaction. Having 5mC-24mer concentration at 10 μM, 10% aqueous DMSO, 100 μM Na₄W₁₀O₂₄, under 365 nm irradiation for 15 minutes, up to 40% of the 5mC residue can be converted to 5fC.

### Labelling

After the 5mC residue in an ODN is converted to 5fC, the ODN can be easily enriched via a bioconjugation reaction at the formyl group with a biotin contained oxyamine or hydrazide (Raiber et al.; Hardisty et al.). This was demonstrated through reacting the obtained mixture with biotinamidohexanoic acid hydrazide (10 mM). The 13mer ODN mixture obtained from photo catalytic oxidation of 5mC-13mer was subjected to bioconjugation reaction. The reaction was performed for 20 hours at 22° C., in the presence of p-anisidine (100 mM) and NH₄OAc (pH=5.0, 40 mM). About 30% of the reaction mixture was found to be conjugated with 1 equiv. of the probe, as confirmed by LC-MS. No detectable amount of conjugation on abasic (AP) site was observed, suggesting that the reaction was selective.

The generated 5fC residue in DNA can alternatively be converted by 1,3-indandione, malononitrile or pyridine-borane (Xia et al.; Zhu et al.; Liu et al.), and used subsequently to introduce a 5fC-to-T mutation during polymerase chain reaction (PCR). A two-step chemical treatment of (i) photochemically oxidising 5mC residue to 5fC in DNA followed malononitrile conversion, and (ii) a PCR to create an overall 5mC-to-T mutation was applied (Scheme 13).

It should be noted that endogenous 5fC exits at very low level compared to 5mC in many genomes, including mammalian genomes (Zhu et al.) and therefore C-to-T mutations obtained by the present method was not expected to originate from false positives due to endogenous 5fC residues. Further, 5fC can be reduced to a 5hmC (Booth et. al.) and protected by a glucose (Song et al.) to prevent false positives during the 5mC-sequencing workflow.

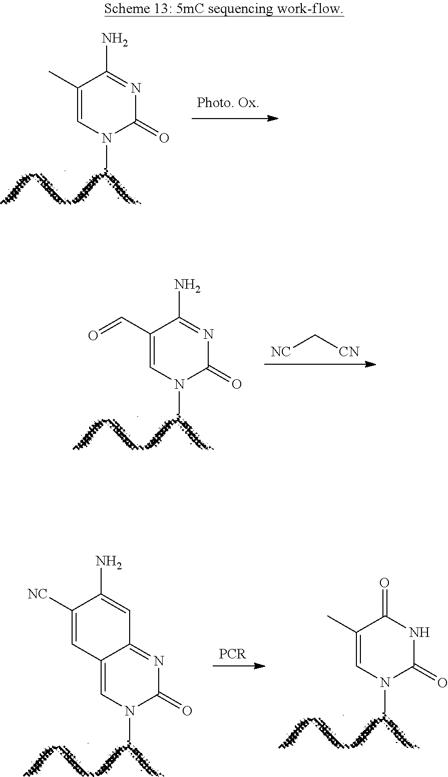

The 5mC-13mer was used to test the two-step chemistry. The oxidised 5mC-13nner was stirred at 22° C. in 400 mM malononitrile aqueous solution for 20 hours. The mass of the corresponding malononitrile adduct was observed in the LC-MS analysis of the purified ODN mixture.

### Sequencing

To demonstrate the method of the present invention can be applied to 5mC-sequencing, a proof-of-the-concept experiment was carried out.

A 100mer single-stranded DNA (ss-DNA) with one 5mC residue (5mC-100mer) was chosen as the target for this study. The 5mC-100mer was treated with the photocatalytic oxidation chemistry followed by a malononitrile conjugation reaction. The obtained ODN mixture was amplified by PCR. A negative control was conducted without photocatalyst. A 100mer ss-DNA containing a 5fC residue instead of 5mC (5fC-100mer) was used as the positive control to directly react with malononitrile. The amplified samples were sequenced.

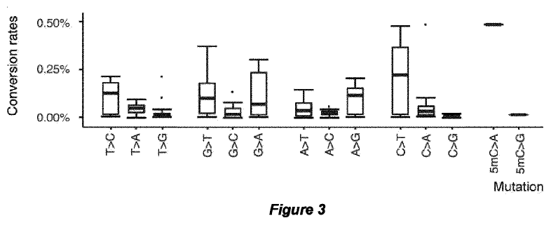

The sequencing data showed up to 32% 5mC-to-T conversion on the target site (FIG. 2). The negative control showed <0.5% 5mC-to-T conversion. The positive control gave 81% 5fC-to-T conversion, indicating that 5fC-to-T conversion is highly efficient. All non-specific mutation rate was found to be below 0.5%, indicating the method is specific towards the 5mC site (FIG. 3).

### References

A number of publications are cited above in order to more fully describe and disclose the invention and the state of the art to which the invention pertains. Full citations for these references are provided below. The entirety of each of these references is incorporated herein.
Bell et al. Genome. Biol. 2019, 20:249
Bilyard et al. Curr Opin. Chem. Bio. 2020, 57, 1
Booth et al., Nat. Chem. 2014, 6, 435
Booth et al. Chem. Rev. 2015, 115, 2240
Breiling et al. Epigenetics Chromatin 2015, 8, 24
DeNizio et al. Biochemistry 2019, 58, 411
Frommer et al. Proc. Natl. Acad. Sci. U. S. A., 1992, 89, 1827
Han et al. Inorg. Chem. 2012, 51, 5118
Hardisty et al., J. Am. Chem. Soc. 2015, 137, 9270
He et al. Science, 2011, 333, 1303
Hong et al. J. Am. Chem. Soc. 2019, 141, 9155
Hu et al. Cell, 2013, 155, 1545
Ito et al. Nature 2010, 466, 1129
Jonasson et al. Chem. Eur. J, 2019, 25, 12091
Jin et al. Adv. Synth. Catal. 2019, 361, 4685
Kriaucionis et al. Science 2009, 324, 929
Lister et al. Science 2013, 341, 1237905
Liu et al. Nat. Biotech. 2019, 37, 424
Lu et al. J. Am. Chem. Soc. 2013, 135, 9315
Lu et at. Chem. Rev. 2015, 115, 2225
Mcinroy et al. Chem. Commun. 2013, 50, 12047
Osberger et al., Nature 2016, 537, 214
Pais et al. Proc. Natl. Acad. Sci. 2015, 112, 4316
Pfaffeneder et al. Angew. Chem. Int. Ed. 2011, 50, 7008
Raiber et al., Genome Biol. 2012, 13, R69
Raiber et al. Nat. Rev. Chem. 2017, 1, 0069
Robertson et al. Nucleic Acids Res. 2011, 39, e55
Sarver et al. Nat. Chem. 2020, 12, 459
Schubeler et al. Nature 2015, 517, 321
Song et al., Nat. Biotechnol. 2011, 29, 68
Tahiliani et al. Science, 2009, 324, 930
White et al. J. Am. Chem. Soc. 2018, 140, 13988
Xia et al., Nat. Methods 2015, 12, 1047
Yu et al. Cell 2012, 149, 1368
Zhu et al. Cell Stem Cell 2017, 20, 720
US 2020/0165661
WO 2019/136413

1. A method of identifying a modified cytosine residue in a sample nucleotide sequence, the method comprising
(i) providing a population of polynucleotides which comprise the sample nucleotide sequence;
(ii) oxidising the modified cytosine residue in the population to form a 5-formylcytosine (5fC) residue through a non-enzymatic, one-electron process;
(iii) labelling the 5-formylcytosine (5fC) residue; and
(iv) identifying the labelled residue within the population,
wherein the modified cytosine residue is selected from a 5-methylcytosine (5mC) residue and a 5-hydroxymethylcytosine (5hmC) residue.

2. The method according to claim 1, wherein step (ii) is performed in the presence of a radical initiator.

3. The method according to claim 2, wherein the radical initiator is a metal-oxo species.

4. The method according to claim 1, wherein in step (ii) the population of polynucleotides is irradiated with light in the presence of a photocatalyst and water.

5. The method according to claim 4, wherein the photocatalyst has an absorbance maximum in the range 300 nm to 600 nm.

6. The method according to claim 4 or claim 5, wherein the photocatalyst is a polyoxometalate.

7. The method according to claim 6, wherein the polyoxometalate comprises tungsten.

8. The method according to claim 4, wherein the photocatalyst is selected from decatungstic acid, phosphotungstic acid, and a salt thereof.

9. The method according to claim 1, wherein step (ii) is performed in the presence of a single-electron oxidant.

10. The method according to claim 9, wherein the single-electron oxidant is an organic single-electron oxidant.

11. The method according to claim 9, wherein the single-electron oxidant is selected from N-fluorobenzenesulfonimide, 5-(trifluoromethyl)dibenzothiophenium tetrafluoroborate, and N-chlorosaccharin.

12. The method according to claim 1, wherein step (iii) comprises converting the 5-formylcytosine (5fC) residue to a uracil analogue, such as by reaction with a nucleophile.

13. The method according to claim 1, wherein step (iii) comprises labelling the 5-formylcytosine (5fC) residue with a detection tag or an isolation tag.

14. The method according to claim 1, wherein step (iii) comprises labelling the 5-formylcytosine (5fC) residue with an isolation tag, such as an isolation tag comprising biotin.

15. The method according to claim 1, wherein step (iii) comprises deaminating the 5-formylcytosine (5fC) residue at the C4 position, and optionally reducing the 5-formylcytosine (5fC) residue, such as reducing the pyrimidine ring.

16. The method according to claim 1, wherein step (iv) comprises the steps of:
(a) sequencing the polynucleotides in the population following step (iii) to produce a treated nucleotide sequence; and
(b) identifying the residue in the treated nucleotide sequence which corresponds to a modified cytosine residue in the sample nucleotide sequence.

17. The method according to claim 1, wherein the modified cytosine residue is a 5-methylcytosine (5mC) residue.

18. A method of oxidising modified cytosine residues in a sample nucleotide sequence, the method comprising;
(i) providing a population of polynucleotides which comprise the sample nucleotide sequence,
(ii) oxidising the modified cytosine residues in the population to form 5-formylcytosine (5fC) residues, wherein the product mole ratio of 5-formylcytosine (5fC) residues to modified cytosine residues is 10:90 or more,
(iii) optionally labelling the 5-formylcytosine (5fC) residues, and
(iv) optionally identifying the labelled 5-formylcytosine (5fC) residues within the population,
wherein the modified cytosine residues are 5-methylcytosine (5mC) residues or 5-hydroxymethylcytosine (5hmC) residues.

19. The method according to claim 18, wherein the product mole ratio of 5-formylcytosine (5fC) residues to 5-hydroxymethylcytosine (5hmC) residues and/or 5-carboxylcytosine (5caC) residues is 2:1 or higher.

20. (canceled)

21. (canceled)

22. (canceled)

23. (canceled)

24. A kit, comprising;
(a) a radical initiator, such as a photocatalyst, such as a polyoxometalate;
(b) a polymerase; and optionally
(c) a single-electron oxidant, such as an organic single-electron oxidant, such as a compound selected from N-fluorobenzenesulfonimide, 5-(trifluoromethyl)dibenzothiophenium tetrafluoroborate, and N-chlorosaccharin.


SEQUENCE LISTING (9 sequences)

SEQ ID NO: 1 - DNA, 13 bp, synthetic construct
  source 1..13: mol_type = other DNA
  modified_base 5: mod_base = OTHER; note = 5-Methylcytosine
acgtcacgtacgt

SEQ ID NO: 2 - DNA, 24 bp, synthetic construct
  source 1..24: mol_type = other DNA
  modified_base 3: mod_base = OTHER; note = 5-Methylcytosine
cgcgttaacgttaacgttaacgcg

SEQ ID NO: 3 - DNA, 100 bp, synthetic construct
  source 1..100: mol_type = other DNA
  modified_base 28: mod_base = OTHER; note = 5-Methylcytosine
tcaggtctggagcaatgcagcagttcgcgcgcatgcttaacagtacagatttcggatccatcgttatcgctctgaaagtacagatcctcagtggttggct

SEQ ID NO: 4 - DNA, 100 bp, synthetic construct; identical to SEQ ID NO: 3
  source 1..100: mol_type = other DNA
  modified_base 28: mod_base = OTHER; note = 5-Formylcytosine

SEQ ID NO: 5 - DNA, 20 bp, synthetic construct
  source 1..20: mol_type = other DNA
agccaaccactgaggatctg

SEQ ID NO: 6 - DNA, 20 bp, synthetic construct
  source 1..20: mol_type = other DNA
tcaggtctggagcaatgcag

SEQ ID NO: 7 - DNA, 13 bp, synthetic construct
  source 1..13: mol_type = other DNA
  modified_base 5: mod_base = OTHER; note = 5-Formylcytosine
acgtcacgtacgt

SEQ ID NO: 8 - DNA, 13 bp, synthetic construct
  source 1..13: mol_type = other DNA
  modified_base 5: mod_base = OTHER; note = (+)-biotinamidohexanoic acid hydrazide conjugate of 5-formylcytosine
acgtcacgtacgt

SEQ ID NO: 9 - DNA, 13 bp, synthetic construct
  source 1..13: mol_type = other DNA
  modified_base 5: mod_base = OTHER; note = Malononitrile conjugate of 5-formylcytosine
acgtcacgtacgt